(19) United States
(12) Patent Application Publication
(10) Pub. No.: US 2005/0272667 A1
(43) Pub. Date: Dec. 8, 2005

(54) Analogs and derivatives of (S,S,R)-(-)-actinonin and uses therefor

(76) Inventors: David Scheinberg, New York, NY (US); William G. Bornmann, Missouri City, TX (US); Francis Sirotnak, New York, NY (US); Howard Scher, Tenefly, NJ (US); Ephraim Vidal, Springfield, NJ (US); Christopher Borella, Cambridge, MA (US)

Correspondence Address: Benjamin Aaron Adler;ADLER & ASSOCIATES, 8011 Candle Lane, Houston, TX, 77071, US

(21) Appl. No.: 11/119,608

(22) Filed: May 2, 2005

Related U.S. Application Data
(63) Continuation-in-part of application No. 10/603,953, filed on Jun. 25, 2003, now Pat. No. 6,887,887.
Application No. 10/603,953 is a division of application No. 10/102,593, filed on Mar. 19, 2002, now Pat. No. 6,660,741.
(60) Provisional application No. 60/277,116, filed on Mar. 19, 2001.

(51) Int. Cl. A61K 38/04; A61K 31/537; A61K 31/445; A61K 31/401

(52) U.S. Cl. 514/19; 514/237.5; 514/317; 514/423; 546/226; 548/530; 544/157

(57) ABSTRACT

The present invention provides analog and derivative compounds of (S,S,R)-(−)-actinonin and methods of asymmetric synthesis thereof having a structure: 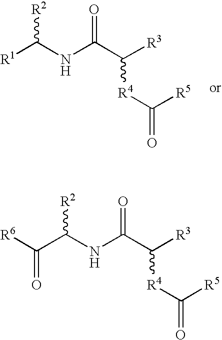
where R¹ is hydrogen, C(O)R⁶ or R¹ in combination with N is 2-oxomorpholine, R² is hydrogen, methyl, CH₂CH(CH₃)₂, (CH₂)₂CH₃, CH(CH₃)₂, (CH₂)₃CH₃, (CH₂)₄NH₂, (CH₂)₃CO₂H, phenyl, 3,4-dichlorophenyl, biphenyl, benzyl, 4-hydroxybenzyl, piperidine, N-Boc-4-piperidine, CH₂-(N-Boc-4-piperidine), 4-tetrahydropyran, CH₂-4-tetrahydropyran, 3-methyl indolyl, 2-naphthyl, 3-pyridyl, 4-pyridyl, 3-thienyl, R³ is R² or C₃₋₈alkyl, R⁴ is C₁₋₃alkyl, R⁵ is NH₂, OH, NHOH, NHOCH₃, N(CH₃)OH, N(CH₃)OCH₃, NHCH₂CH₃, NH(CH₂CH₃), NHCH₂(2,4-(OCH3)₂Ph, NHCH₂(4-NO₂)Ph, NHN(CH₃)₂, proline, or 2-hydroxymethyl pyrrolidine and R⁶ is an optionally substituted or halogenated alkyl, aryl, heteroalkyl or heteroaryl amine where R⁶ further comprising a cyclic or bicyclic structure. Also provided are methods for treating a neoplastic disease or for inhibiting tumor cell growth using the compounds present invention or using the compound (S,S,R)-(−)-actinonin.

### CROSS-REFERENCE TO RELATED APPLICATIONS

This application is a continuation-in-part of U.S. Ser. No. 10/603,953, filed Jun. 25, 2003, which is a divisional of non-provisional application U.S. Ser. No. 10/102,593, filed Mar. 19, 2002, issued as U.S. Pat. No. 6,660,741 on Dec. 9, 2003, which claims benefit of priority of provisional U.S. Ser. No. 60/277,116, filed Mar. 19, 2001, now abandoned.

### BACKGROUND OF THE INVENTION

1. Field of the Invention

The present invention relates generally to the fields of organic chemistry and anti-tumor compounds. More specifically, the present invention relates to the asymmetric synthesis of (S,R.R)-(−)-actinonin and derivatives and analogs thereof and their uses as anti-tumor agents.

2. Description of the Related Art

(S,S,R)-(−)-Actinonin (1), was first isolated by Green and Singh from the Malayan strain of Actinomycete, Streptomyces sp. Cutter 12 (N.C.I.B. 8845) (FIG. 1). It has been shown that actinonin exhibits antibiotic and anti-tumor properties (1-7). Studies have demonstrated that actinonin exhibits cytotoxicity towards tumor cell lines in vitro (4). Furthermore, actinonin induces G₁ arrest and apoptosis in human leukemia and lymphoma cells. It also treats AKR leukemia in AKR mice with minimal toxicity.

Although actinonin is commercially available and usually extracted from Actinomycete and Streptomyces bacteria, specifically Streptomyces roseopallidus (9, 10), the yield of compound derived is miniscule. For example, out of a typical ten day culture that yields eleven liters of filtrate, only 146 mg of pure actinonin are isolated. Currently, actinonin is synthesized by either of two synthetic schema. In Ollis' 1975 synthetic method, the synthesis of actinonin is non-stereoselective and the diastereomers have to be separated; difficult process producing small yields (11). Davies' 1992 synthesis is stereoselective and represents the first asymmetric synthesis of (−)-actinonin. An Fe(II)-based chiral auxiliary is used to introduce chirality at the α-position of a carboxylic acid (13,14). However, this process causes disposal problems and therefore commercialization of the synthetic (−)-actinonin is doubtful. It is therefore necessary to develop a method for multi-gram synthesis of actinonin for further testing in various cancer cell lines and for animal studies.

The prior art is deficient in the lack of effective means of asymmetically synthesizing (S,R,R)-(−)-actinonin, its derivatives and its analogs for use as anti-tumor agents. The present invention fulfills this long-standing need and desire in the art.

### SUMMARY OF THE INVENTION

The present invention is directed to an analog or derivative compound of (S,S,R)-(−)-actinonin having the structure: 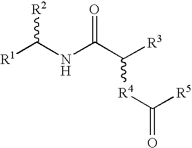
R¹ is hydrogen, C(O)R⁶ or R¹ in combination with N is 2-oxomorpholine. R² is hydrogen, methyl, CH₂CH(CH₃)₂, (CH₂)₂CH₃, CH(CH₃)₂, (CH₂)₃CH₃, (CH₂)₄NH₂, (CH₂)₃CO₂H, phenyl, 3,4-dichlorophenyl, biphenyl, benzyl, 4-hydroxybenzyl, piperidine, N-Boc-4-piperidine, CH₂—(N-Boc-4-piperidine), 4-tetrahydropyran, CH₂-4-tetrahydropyran, 3-methyl indolyl, 2-naphthyl, 3-pyridyl, 4-pyridyl, 3-thienyl. R³ is R² or C₃₋₈alkyl and R⁴ is C₁₋₃alkyl. R⁵ is NH₂, OH, NHOH, NHOCH₃, N(CH₃)OH, N(CH₃)OCH₃, NHCH₂CH₃, NH(CH₂CH₃), NHCH₂(2,4-(OCH3)₂Ph, NHCH₂(4-NO₂)Ph, NHN(CH₃)₂, proline, or 2-hydroxymethyl pyrrolidine and R⁶ is an optionally substituted or halogenated alkyl, aryl, heteroalkyl or heteroaryl amine where R⁶ further comprising a cyclic or bicyclic structure. Substituted compounds may comprise the substituents in Table 1.

The present invention is directed to a related compound having the structure: 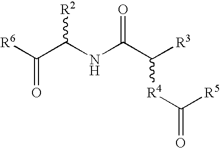
where R⁶ is NHCH₂Ph, NHCH₃, NHCH₂CH₃, N(CH₃)₂, N(CH₂CH₃)₂, NHCH₂(2,4-(OCH₃)₂Ph, NHCH₂(4-NO₂Ph), hexamethyleneamine, hexamethyleneimine, methyl 2- or 3-hexamethyleneamine carboxylate, heptamethyleneamine, pyrrole, indole, aziradine, imidazole, 1,4-dioxan-2-yl-methylamine, 3,4-dihydro-2H-1,4-benzoxazin-6-ol, 6-methoxy-1,2,3,4-tetrahydro-isoquinoline, piperazin-1-yl-pyridin-3-yl-methanone or optionally substituted pyrrolidine, piperidine, piperazine, morpholine, indoline, proline, or azetidine or pharmaceutically acceptable salts or hydrates thereof.

The present invention also is directed to a method for asymmetrically synthesizing the compounds described herein generally comprising synthesizing and coupling an O-protected R⁵ or the chloride thereof with an O-protected R⁶-1-carbonyl-C2(R²)-carbamoyl-methylene(R³)—R⁴-carboxylic acid 2,5-dioxo-pyrrolidin-1-yl ester to form an R⁶-1-carbonyl-C2(R²)-carbamoyl-methylene(R³)—R⁴-carbonyl-R⁵ where R⁶ and R⁵ are O-protected. R⁶ and R⁵ are hydrogenated with hydrogen gas thereby synthesizing the chemical compound.

The present invention is directed further to methods of treating a neoplastic disease in an individual. The method comprises administering a pharmacologically effective amount of (S,S,R)-(−)-actinonin or of the analog or derivative compound described herein. The present invention is directed to related methods of inhibiting the growth of a tumor cell by contacting the cell with a pharmacologically effective dose of (S,S,R)-(−)-actinonin or other chemical compound disclosed herein.

Other and further aspects, features, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention are briefly summarized above may be had by reference to certain embodiments thereof which are illustrated in the appended drawings. These drawings form a part of the specification. It is to be noted; however, that the appended drawings illustrate preferred embodiments of the invention and therefore are not to be considered limiting in their scope.

FIG. 1A depicts the structure of (S,S,R)-(−)-actinonin (1).

FIG. 1B depicts structurally how (S,S,R)-(−)-actinonin (1) is divided into fragments A, B and C

FIG. 1C depicts the structure of Evan's chiral auxiliary (2).

FIG. 2 depicts an alternative synthetic scheme for synthesizing and adding fragments A and C to fragment B for compounds having a benzyl functional group at R² (Scheme 1).

FIG. 3A depicts the synthetic scheme for the α-pentylsuccinate fragment B (Scheme 2).

FIG. 3B depicts the synthetic scheme for the pseudodipeptide derivative of L-prolinol and L-valine fragment A (Scheme 3)

FIG. 3C depicts the synthetic scheme for the joining of fragments A, B and C to yield (S,S,R)-(−)-actinonin (Scheme 4).

FIG. 4A shows the synthetic scheme of the actinonin analogs N4-hydroxy-N1-(1-(2-methyl-pyrrolidine-1-carbonyl)-3-methyl-propyl)-2-pentyl-succinamide (27) (Scheme 5).

FIG. 4B shows the synthetic sequence of the actinonin analogs N4-hydroxy-N1-(1-(2-hydroxymethyl-pyrrolidine-1-carbonyl)-3-methyl-butyl)-2-pentyl-succinamide (35) (Scheme 6).

### DETAILED DESCRIPTION OF THE INVENTION

In one embodiment of the present invention there is provided an analog or derivative compound of (S,S,R)-(−)-actinonin having the structure: 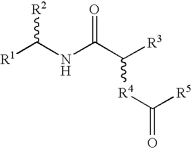
where R¹ is hydrogen, C(O)R⁶ or R¹ in combination with N is 2-oxomorpholine; R² is hydrogen, methyl, CH₂CH(CH₃)₂, (CH₂)₂CH₃, CH(CH₃)₂, (CH₂)₃CH₃, (CH₂)₄NH₂, (CH₂)₃CO₂H, phenyl, 3,4-dichlorophenyl, biphenyl, benzyl, 4-hydroxybenzyl, piperidine, N-Boc-4-piperidine, CH₂-(N-Boc-4-piperidine), 4-tetrahydropyran, CH₂-4-tetrahydropyran, 3-methyl indolyl, 2-naphthyl, 3-pyridyl, 4-pyridyl, 3-thienyl; R³ is R² or C₃₋₈alkyl, R⁴ is C₁₋₃alkyl; R⁵ is NH₂, OH, NHOH, NHOCH₃, N(CH₃)OH, N(CH₃)OCH₃, NHCH₂CH₃, NH(CH₂CH₃), NHCH₂(2,4-(OCH3)₂Ph, NHCH₂(4-NO₂)Ph, NHN(CH₃)₂, proline, or 2-hydroxymethyl pyrrolidine; and R⁶ is an optionally substituted or halogenated alkyl, aryl, heteroalkyl or heteroaryl amine, said R⁶ further comprising a cyclic or bicyclic structure; or pharmaceutically acceptable salts or hydrates thereof.

In one aspect of this embodiment R¹ is hydrogen, R² is (CH₂)₃CH₃, R³ is pentyl, R⁴ is methylene, and R⁵ is NHOH. The derivative compound may be N4-hydroxy-N1-butyl-2-pentylsuccinamide.

In another aspect R¹ in combination with N is 2-oxomorpholine, R² is CH(CH₃)₂, R³ is pentyl, R⁴ is methylene, and R⁵ is NHOH. The derivative compound may be N-hydroxy-3-(4-isopropyl-2-oxooxazolidine-3-carbonyl)octanamide.

In yet another aspect of this embodiment the analog or derivative compound has the structure: 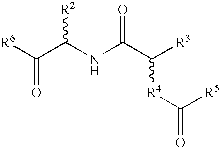
R² is hydrogen, methyl, CH₂CH(CH₃)₂, (CH₂)₂CH₃, CH(CH₃)₂, (CH₂)₃CH₃, (CH₂)₄NH₂, (CH₂)₃CO₂H, phenyl, 3,4-dichlorophenyl, biphenyl, benzyl, 4-hydroxybenzyl, piperidine, N-Boc-4-piperidine, CH₂-(N-Boc-4-piperidine), 4-tetrahydropyran, CH₂-4-tetrahydropyran, 3-methyl indolyl, 2-naphthyl, 3-pyridyl, 4-pyridyl, 3-thienyl; R³ is R² or C₃₋₈alkyl, R⁴ is C₁₋₃ alkyl; R⁵ is NH₂, OH, NHOH, NHOCH₃, N(CH₃)OH, N(CH₃)OCH₃, NHCH₂CH₃, NH(CH₂CH₃), NHCH₂(2,4-(OCH3)₂Ph, NHCH₂(4-NO₂)Ph, NHN(CH₃)₂, proline, or 2-hydroxymethyl pyrrolidine; and R⁶ is NHCH₂Ph, NHCH₃, NHCH₂CH₃, N(CH₃)₂, N(CH₂CH₃)₂, NHCH₂(2,4-(OCH₃)₂Ph, NHCH₂(4-NO₂Ph), hexamethyleneamine, hexamethyleneimine, methyl 2- or 3-hexamethyleneamine carboxylate, heptamethyleneamine, pyrrole, indole, aziradine, imidazole, 1,4-dioxan-2-yl-methylamine, 3,4-dihydro-2H-1,4-benzoxazin-6-ol, 6-methoxy-1,2,3,4-tetrahydro-isoquinoline, piperazin-1-yl-pyridin-3-yl-methanone. Additionally in this aspect R⁶ may be pyrrolidine optionally substituted with 2-methylamino, 2-hydroxycarbamoyl, one of 2- or 3-hydroxymethyl, one of 2- or 3-methyl, ethyl, benzyl or phenyl, one of 2,3-, 2,4-, or 2,5-dimethyl, 2,5-diethyl, one of methyl-, ethyl-, t-butyl- or benzyl-3-carboxylate, or methyl-(2-methyl-5-carboxylate). R⁶ may be piperidine optionally substituted with 2- or 3-methyl or ethyl, one of methyl-, ethyl-, or benzyl-2-, 3-, 4-carboxylate. R⁶ may be piperazine optionally substituted with 1-benzyl, N-t-boc, 1-furfuryl, 1-isonicotinoyl, or -one of pyridin-2-, 3- or 4-ylmethyl. R⁶ may be morpholine optionally substituted with one of methyl-, ethyl-, or benzyl-2- or 3-carboxylate. R⁶ may be indoline optionally substituted with one of C2-C7 fluoro or methyl-2-carboxylate. R⁶ may be proline optionally substituted to independently form a methyl, ethyl, benzyl or t-butyl ester or R⁶ may be azetidine optionally substituted with one of 2- or 3-methyl or ethyl or a methyl-, ethyl- or benzyl-2- or 3-carboxylate.

In this aspect R² may be hydrogen, (CH₂)₃CH₃, or (CH₂)₃CO₂H, R³ may be pentyl, R⁴ may be methylene, R⁵ may be NHOH and R⁶ may be piperidine. Representative examples of analog compounds are N4-hydroxy-N1-(2-oxo-2-(piperidin-1-yl)ethyl)-2-pentyl succinamide, 4-((2-(2(hydroxyamino)-2-oxoethyl)heptanamido)-5-oxo-5-(piperidin-1-yl) pentanoic acid or N4-hydroxy-N1-(1-piperidine-1-carbonyl)-pentyl)-2-pentyl succinamide. Also in this aspect R² may be CH(CH₃)₂, R³ may be pentyl, R⁴ is methylene, R⁵ may be NHOH and R⁶ may be morpholine, hexamethyleneimine, or NH(CH₂CH₃). Representative examples of analog compounds are N4-hydroxy-N1N1-(1-diethylamino-1-carbonyl)-2-methyl-propyl)-2-pentyl succinamide, N4-hydroxy-N1-(1-piperidine-1-carbonyl)-2-methyl-propyl)-2-pentyl succinamide or N-hydroxy-3-(4-isopropyl-2-oxooxazolidine-3-carbonyl)octanamide. Furthermore, in this aspect R² in combination with N is pyrrolidine. A representative example of an analog compound is N-hydroxy-3(2-(piperidine-1-carbonyl)pyrrolidine-1-carbonyl)octanamide. Further yet in this aspect R⁵ and R⁶ combine to form a ring. A representative example of a cyclic analog compound is (5R,8S,12aS)-8-benzyl-5-pentyloctahydropyrrolo[2,1-c][1,4,7] oxadiaza cycloundecine-3,6,9(1H)-trione.

In another embodiment of this invention there is provided method for asymmetrically synthesizing a chemical compound having the structures described herein, comprising the steps of forming an optionally O-protected R⁶-1-carbonyl-C2-(R²)-methyleneamine from R⁶ and an N-protected, optionally O-protected, R²-amino acid 2,5-dioxo-pyrrolidinyl ester and deprotecting said N-protected R²-amino acid with a suitable agent comprising trifluoroacetic acid; forming an R³-carbonyl-oxazolidone from 4-isopropyl-oxazolidin-2-one and R³-carbonyl chloride; treating a solution of 4-(S)-isopropyl-oxazolidin-2-one with a solution of a base comprising n-butyl lithium in hexanes and adding an R³-carbonyl chloride thereby forming an R³-carbonyl oxazolidinone; treating a solution of the R³-carbonyl oxazolidinone sequentially with a base comprising lithium diisopropylamide and with a bromo-R⁴ acid-tert-butyl ester thereby forming an oxazolidine-R³-carbonyl-R⁴-acid tert-butyl ester; treating a mixture of the an oxazolidine-R³-carbonyl-R⁴-acid tert-butyl ester in tetrahydrofuran and water sequentially with hydrogen peroxide in water and with lithium hydroxide in water thereby forming a C2(R³)—R⁴-dicarboxylic acid tert-butyl ester; treating a mixture of the C2(R³)—R⁴-dicarboxylic acid 4-tert-butyl ester and hydroxysuccinimide in a solvent comprising dioxane or dimethylformamide with an imide comprising dicyclohexylcarbodiimide thereby forming an C2(R³)—R⁴-dicarboxylic acid tert-butyl ester-(2,5-dioxo-pyrrolidin-1-yl) ester; treating a solution of the optionally O-protected R¹-1-carbonyl-2C(R²)-methyleneamine in a solvent comprising tetrahydrofuran sequentially with triethylamine and with the C2(R³)—R⁴-dicarboxylic acid tert-butyl ester-(2,5-dioxo-pyrrolidin-1-yl) ester thereby forming an optionally O-protected R¹-1-carbonyl-C2-(R²)-carbamoyl-methylene(R³)—R⁴-carboxylic acid tert-butyl ester; treating a solution of said optionally O-protected R¹-1-carbonyl-C2(R²)-carbamoyl-methylene(R³)—R⁴-carboxylic acid tert-butyl ester in a solvent comprising methylene chloride with trifluoroacetic acid thereby forming an optionally O-protected R¹-1-carbonyl-C2(R²)-carbamoyl-methylene(R³)—R⁴-carboxylic acid; treating the optionally O-protected R¹-1-carbonyl-C2-(R²)-carbamoyl-methylene(R³)—R⁴-carboxylic acid and hydroxysuccinamide with an imide comprising dicyclohexylcarbodiimide thereby forming a optionally O-protected R¹-1-carbonyl-C2(R²)-carbamoyl-methylene(R³)—R⁴-carboxylic acid 2,5-dioxo-pyrrolidin-1-yl ester; treating a suspension of R⁵ or the chloride thereof, where R⁵ is optionally O-protected, in a solvent comprising dimethylformamide sequentially with triethylamine and with a solution of the O-protected R¹-1-carbonyl-C2(R²)-carbamoyl-methylene(R³)—R⁴-carboxylic acid 2,5-dioxo-pyrrolidin-1-yl ester in a solvent comprising dimethylformamide thereby forming an R¹-1-carbonyl-C2(R²)-carbamoyl-methylene(R³)—R⁴-carbonyl-R⁵, where R¹ and R⁵ are independently optionally O-protected; and hydrogenating R¹ and R⁵, where R¹ and R⁵ independently comprise an O-protecting group, with hydrogen gas and a catalyst comprising palladium hydroxide in activated carbon where the analog compounds disclosed herein are formed.

In one aspect of this embodiment R² may be hydrogen, CH₂CH₃, (CH₂)₃CH₃, CH(CH₃)₂, or (CH₂)₃CO₂H; R³ is pentyl; R⁴ is methylene; R⁵ may be NH₂, OH, NHOH, NHOCH₃, N(CH₃)OH, N(CH₃)OCH₃, NHCH₂CH₃, NH(CH₂CH₃), NHCH₂(2,4-(OCH3)₂Ph, NHCH₂(4-NO₂)Ph, NHN(CH₃)₂, proline, 2-hydroxymethyl pyrrolidine. piperidine or 1-methyl-piperazine; and R⁶ may be piperidine, morpholine, hexamethyleneimine, or NH(CH₂CH₃). Representative examples of analog or derivative compounds synthesized by the method disclosed herein are N4-hydroxy-N1-(2-oxo-2-(piperidin-1-yl)ethyl)-2-pentyl succinamide, 4-((2-(2(hydroxyamino)-2-oxoethyl)heptanamido)-5-oxo-5-(piperidin-1-yl) pentanoic acid, N4-hydroxy-N1-(1-piperidine-1-carbonyl)-pentyl)-2-pentyl succinamide, N4-hydroxy-N1,N1-(1-diethylamino-1-carbonyl)-2-methyl-propyl)-2-pentyl succinamide, N4-hydroxy-N1-(1-piperidine-1-carbonyl)-2-methyl-propyl)-2-pentyl succinamide, N4-hydroxy-N1-(1-morpholine-1-carbonyl)-2-methyl-propyl))-2-pentyl succinamide, N-hydroxy-3(2-(piperidine-1-carbonyl)pyrrolidine-1-carbonyl) octanamide, (5R,8S,12aS)-8-benzyl-5-pentyloctahydropyrrolo[2,1-c][1,4,7]oxadiazacyclundecine-3,6,9(1H)-trione.

In yet another embodiment of the present invention there is provided a method for the treatment of a neoplastic disease in an individual comprising administering a pharmacologically effective amount of (S,S,R)-(−)-actinonin or the analog or derivative compounds disclosed herein or a pharmaceutically acceptable salt or hydrate thereof.

Representative examples of analog or derivative compounds used for treatment are N4-hydroxy-N1-(1-(2-hydroxymethyl-pyrrolidine-1-carbonyl)-3-methyl-butyl)-2-pentyl-succinamide, N1-(1-(2-methyl-pyrrolidine-1-carbonyl)-2-methyl-propyl)-2-pentyl-succinamide, N1-(1-benzyl-2-(2-hydroxymethyl-pyrrolidin-1-yl)-2-oxo-ethyl)-N4-hydroxy-2-pentyl-succinamide, N4-hydroxy-N1-(1-(2-hydroxymethyl-pyrrolidine-1-carbonyl)-2-methyl-propyl)-2-methyl-succinamide, N4-hydroxy-N1-(1-benzyl-2-(2-methyl-pyrrolidin-1-yl)-2-oxo-ethyl-2-pentyl-succinamide, N4-hydroxy-N1-(1-(methyl-2-carboxy-pyrrolidine-1-carbonyl)-2-methyl-propyl)-2-pentyl-succinamide, N4-hydroxy-N1-(2-oxo-2-(piperidin-1-yl)ethyl)-2-pentyl succinamide, 4-((2-(2(hydroxyamino)-2-oxoethyl)heptanamido)-5-oxo-5-(piperidin-1-yl) pentanoic acid, N4-hydroxy-N1-(1-piperidine-1-carbonyl)-pentyl)-2-pentyl succinamide, N4-hydroxy-N1,N1-(1-diethylamino-1-carbonyl)-2-methyl-propyl)-2-pentyl succinamide, N4-hydroxy-N1-(1-piperidine-1-carbonyl)-2-methyl-propyl)-2-pentyl succinamide, or N4-hydroxy-N1-(1-morpholine-1-carbonyl)-2-methyl-propyl))-2-pentyl succinamide, N-hydroxy-3(2-(piperidine-1-carbonyl)pyrrolidine-1-carbonyl)octanamide, (5R,8S,12aS)-8-benzyl-5-pentyloctahydropyrrolo[2,1-c][1,4,7]oxadiazacyclundecine-3,6,9(1H)-trione, N4-hydroxy-N1-butyl-2-pentylsuccinamide, or N-hydroxy-3-(4-isopropyl-2-oxooxazolidine-3-carbonyl)octanamide.

Representative examples of a neoplastic disease are a human ovarian carcinoma, a prostate carcinoma, a mammary carcinoma, a head and neck squamous cell carcinoma, a non-small-cell-lung-cancer adenocarcinoma, non-small-cell-lung-cancer squamous cell carcinoma, or acute myologenous leukemia.

In a related embodiment of the present invention there is provided a method for inhibiting the growth of a tumor cell comprising administering a pharmacologically effective amount of (S,S,R)-(−)-actinonin or the analog or derivative compounds disclosed herein or a pharmaceutically acceptable salt or hydrate thereof. The analog or derivative compounds are as described supra. The tumor cells may comprise those neoplastic diseases described supra.

The following definitions are given for the purpose of facilitating understanding of the inventions disclosed herein. Any terms not specifically defined should be interpreted according to the common meaning of the term in the art.

As used herein, the term, “a” or “an” may mean one or more. As used herein in the claim(s), when used in conjunction with the word “comprising”, the words “a” or “an” may mean one or more than one. As used herein “another” may mean at least a second or more.

As used herein, the term “alkyl” shall refer to optionally substituted straight, branched or cyclic hydrocarbon chains.

As used herein, the term “aryl” shall refer to optionally substituted aromatic mono- or bicyclic hydrocarbons.

As used herein, the term “heteroatom” or “heterocyclic” refers to an atom in an organic molecule or compound that is nitrogen, oxygen, sulfur, phosphorus or a halogen or an alkyl, aryl or aromatic compound comprising the heteroatom. It is particularly contemplated that a heteroatom is nitrogen, oxygen or sulfur.

As used herein, the term “contacting” refers to any suitable method of bringing one or more of the compounds described herein into contact with a cell. In vitro or ex vivo this is achieved by exposing the cells to the compound in a suitable medium. For in vivo applications, any known method of administration is suitable as described herein.

As used herein, the term “individual” shall refer to animals and humans.

As used herein, the terms “neoplastic” “tumor” or “tumor cell” shall refer to a mass of tissue or cells characterized by, inter alia, abnormal cell proliferation. The abnormal cell proliferation results in growth of these tissues or cells that exceeds and is uncoordinated with that of the normal tissues or cells and persists in the same excessive manner after the stimuli which evoked the change ceases or is removed. Neoplastic tissues or cells show a lack of structural organization and coordination relative to normal tissues or cells which usually results in a mass of tissues or cells which can be either benign or malignant. As would be apparent to one of ordinary skill in the art, the term “cancer” refers to a malignant neoplasm.

As used herein, the term “inhibiting” or “inhibition” of the growth of proliferating tumor cells or neoplastic cells shall include partial or total growth inhibition and also is meant to include decreases in the rate of proliferation or growth of the cells. The biologically inhibitory dose of the composition of the present invention may be determined by assessing the effects of the test element on target malignant or abnormally proliferating cell growth in tissue culture, tumor growth in animals and cell culture or any other method known to those of ordinary skill in the art.

The following abbreviations may be used herein: THF: tetrahydrofuran; DMF: dimethylformamide; TFA: trifluoroacetic acid; n-BuLi: n-butyl lithium; OHsuccinNH: hydroxysuccinamide; HOBT: 1-hydroxybenzotriazole; EDC: 1-(3-dimethylaminopropyl)-3-ethylcarbodiimide hydrochloride; DCC: dicyclohexylcarbodiimide; CH₂Cl₂: methylene chloride; and TLC: thin layer chromatography; HNSSC: head and neck squamous cell carcinoma: NSCLC-AdCa: non-small-cell-lung-cancer adenocarcinoma: NSCLS-SSC: non-small-cell-lung-cancer squamous cell carcinoma; and AML: acute mylegenous leukemia.

Thus the present invention is directed toward the synthesis and characterization of novel agents that inhibit tumor growth in vitro and in vivo. More specifically, this invention provides for the asymmetric synthesis and effective use of (S,S,R)-(−)-actinonin (1) and its analogs and derivatives as inhibitors of tumor growth in neoplastic diseases. The novel (S,S,R)-actinonin analogs and derivatives of the present invention have the general formula shown below:

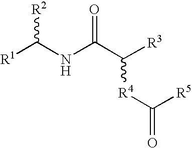

Generally, the compounds are substituted succinamides. R1 may be hydrogen, C(O)R⁶ or R¹ in combination with N is 2-oxomorpholine. Preferably, R¹ is R⁶ where R⁶ is an optionally substituted or halogenated alkyl, aryl, heteroalkyl or heteroaryl amine and usually comprises a cyclic or bicyclic structure. R² and R³ are primarily alkyl although these sidechains may be heterocycles, e.g., piperidinyl, napthyl, pyridyl, or thienyl. Preferably R² is a straight or branched C₂₋₄ alkyl and R3 is R² or C₃₋₈alkyl. R² may be a carboxylic acid moiety. More preferably, R² is a branched C₂₋₃ alkyl and R³ is pentyl. Alternatively, R² in combination with N in the generic structure form a heterocyclic amine such as, but not limited to pyrrolidine. R⁴ is preferably a methylene group although dicarboxylic acids where R⁴ is either ethylene or propylene may be synthesized. The amide functional group R⁵ may be mono- or di-substituted with a C₁₋₂-alkyl, -alkoxy or phenoxy group(s), preferably R⁵ is hydroxyamine. It is contemplated that R5 and R6 may link to form a ring structure. For example, hydroxy moieties on R5 and R6 may link.

Preferably, (S,S,R)-actinonin analogs have the structure shown below:

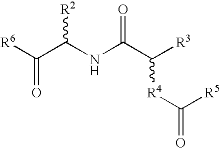

In the present invention R⁶ may be a mono- or di-substituted simple straight chain or cyclic amine or imine such as methyl, ethyl or benzyl amine or optionally substituted proline, azetidine, aziradine, or hexa- or heptamethyleneamime or hexa- or heptamethyleneimine. Generally R⁶ is a heterocycle optionally substituted in the C2 or C3 positions on the ring. Preferably the heterocycle is pyrrolidine, but may also be pyrrole, indole, indoline, morpholine, piperidine, isoquinoline, or piperazine. Ring substitutions at the C2 or C3 positions can be methyl, ethyl, benzyl, or the heterocylic esters thereof with hydroxymethyl, methyl or hydroxycarbamoyl the preferred substituent on the pyrrolidine ring. Additionally the heterocycles may be substituted with other heterocycles such as pyridinyl, isonicotinyl or furfuryl rings. The heterocycles may be halogenated on any of the available ring positions with a representative example being the fluoroindolines. The substituents disclosed for these R²-R⁶ groups are not limited to these groups and are not in any way limiting to the scope of the invention. Table I details the R⁶ group substituents for these actinonin analogs.

TABLE 1
R⁶ substituents for analogs/derivatives of (S,S,R)-(−)-actinonin

| | Compound substituents | | |
|---|---|---|---|
| | | ester | |
| Compound | alkyl/aryl | (carboxylate) | Misc. |
| pyrrole | | | |
| indole | | | |
| aziradine | | | |
| imidazole | | | |
| proline | | methyl | |
| | | ethyl | |
| | | t-butyl | |
| azetidine | 2 or 3-methyl | 2 or 3-methyl | |
| | 2 or 3-ethyl | 2 or 3-ethyl | |
| | | 2 or 3-t-butyl | |
| indoline | | methyl-2 | 2-7 Fl |
| pyrrolidine | 2 or 3-methyl | methyl-2 | 2-NHCH3 |
| | 2 or 3-ethyl | ethyl-2 | 2 or 3-NHOH |
| | 2 or 3-t-butyl | t-butyl-2 | 2-OHcarbamoyl |
| | 2 or 3-phenyl | benzyl-2 | |
| | 2,3-dimethyl | methyl-2-methyl-5 | |
| | 2,4-dimethyl | | |
| | 2,5-dimethyl | | |
| | 2,5-diethyl | | |
| piperidine | 2 or 3-methyl | methyl-2, -3 or -4 | |
| | 2 or 3-ethyl | ethyl-2, -3 or -4 | |
| | | benzyl-2, -3 or -4 | |
| morpholine | | methyl-2 or -3 | |
| | | ethyl-2 or -3 | |
| | | benzyl-2 or -3 | |
| piperazine | 1-benzyl | | 1-furfuryl |
| | | | 1-isonicotinoyl |
| | | | N-tBoc |
| | | | pyridin-2yl-methyl |
| | | | pyridin-3yl-methyl |
| | | | pyridin-4yl-methyl |
| hexamethyleneamine | | methyl-2 or -3 | |
| heptamethyleneamine | | | |
| NH(R) or NRR | methyl | | |
| | ethyl | | |
| | benzyl | | |
| | dimethyl | | |
| | diethyl | | |
| | 2,4-dimethoxy benzyl | | |
| | 4-nitrobenzyl | | |
| 1,4-dioxan-2-yl- | | | |
| methylamine | | | |
| 3,4-dihydro-2H- | | | |
| 1,4-benzoxazin-6-ol | | | |
| 6-methoxy-1,2,3,4- | | | |
| tetrahydro-isoquinoline | | | |
| piperazin-1-yl-pyridin- | | | |
| 3-yl-methanone | | | |

Analog and derivative compounds of the present invention may be, but are not limited to, N4-hydroxy-N1-(1-(2-methyl-pyrrolidine-1-carbonyl)-3-methyl-propyl)-2-pentyl-succinamide (27), N4-hydroxy-N1-(1-(2-hydroxymethyl-pyrrolidine-1-carbonyl)-3-methyl-butyl)-2-pentyl-succinamide (35), N4-hydroxy-N1-(1-(2-hydroxymethyl-pyrrolidine-1-carbonyl)-3-methyl-butyl)-2-pentyl succinamide (41), N1-(1-benzyl-2-(2-hydroxymethyl-pyrrolidin-1-yl)-2-oxo-ethyl)-N4-hydroxy-2-pentyl-succinamide (42), N4-hydroxy-N1-(1-(4-hydroxy-benzyl)-2-(2-hydroxymethyl-pyrrolidin-1-yl)-2-oxo-ethyl)-2-pentyl-succinamide (43), N4-hydroxy-N1-(2-(2-hydroxymethyl-pyrrolidin-1-yl)-1(1H-indol-3-yl-methyl)-2-oxo-ethyl)-2-pentyl-succinamide (44), N1-(5-amino-1-(2-hydroxymethyl-pyrrolidine-1-carbonyl)-pentyl)-N4-hydroxy-2pentyl-succinamide (45), N4-hydroxy-N1-(1-(2-hydroxymethyl-piperidine-1-carbonyl)-2-methyl-propyl)-2-pentyl-succinamide (46), N4-hydroxy-N1-(1-(2-hydroxycarbamoyl-pyrrolidine-1-carbonyl)-3-methyl-butyl)-2-pentyl succinamide (47), N4-hydroxy-N1-(1-(2-hydroxymethyl-pyrrolidine-1-carbonyl)-2-methyl-propyl)-2-methyl-succinamide (48), N1-(1-(2-hydroxymethyl-pyrrolidine-1-carbonyl)-3-methyl-butyl)-2-pentyl-succinamide (49), and N1-(1-benzyl-2-(2-hydroxymethyl-pyrrolidin-1-yl)-2-oxo-ethyl)-2-pentyl-succinamide (50), N1-(1-(2-methyl-pyrrolidine-1-carbonyl)-2-methyl-propyl)-2-pentyl-succinamide (51), N4-hydroxy-N1-(1-benzyl-2-(2-methyl-pyrrolidin-1-yl)-2-oxo-ethyl-2-pentyl-succinamide (52), N4-hydroxy-N1-(1-(2-methylamine-pyrrolidine-1-carbonyl)-2-methyl-propyl)-2-pentyl-succinamide (53), 3-[1-(2-hydroxymethyl-pyrrolidin-1-yl)-2-benzylcarbamoyl]-octanoic acid (54), N4-hydroxy-N1-(1-(methyl-2-carboxy-pyrrolidine-1-carbonyl)-2-methyl-propyl)-2-pentyl-succinamide (55), N4-hydroxy-N1-(1-(2-carboxy-pyrrolidine-1-carbonyl)-2-methyl-propyl)-2-pentyl-succinamide (56), N4, N4-diethyl-N1-(1-benzyl-2-(2-hydroxymethyl-pyrrolidin-1-yl)-2-oxo-ethyl-2-pentyl-succinamide (57), N4-ethyl-N1-(1-benzyl-2-(2-hydroxymethyl-pyrrolidin-1-yl)-2-oxo-ethyl-2-pentyl-succinamide (58), N4-(2,4-methoxybenzyl)-N1-(1-benzyl-2-(2-hydroxymethyl-pyrrolidin-1-yl)-2-oxo-ethyl-2-pentyl-succinamide (59), 2-(N′,N′-dimethyl-hydrazinocarbonylmethyl)-heptanoic acid [1-benzyl-2-(2-hydroxymethyl-pyrrolidin-1-yl)-2-oxo-ethyl]-amide (60), N4-(4-nitrobenzyl)-N1-(1-benzyl-2-(2-hydroxymethyl-pyrrolidin-1-yl)-2-oxo-ethyl-2-pentyl-succinamide (61), 2-[2-(4-methyl-piperazin-1-yl)-2-oxo-ethyl]-heptanoic acid [1-benzyl-2-(2-hydroxymethyl-pyrrolidin-1-yl)-2-oxo-ethyl]-amide (62), N4-(methoxy)-N1-(1-benzyl-2-(2-hydroxymethyl-pyrrolidin-1-yl)-2-oxo-ethyl-2-pentyl-succinamide (63), N4-(piperidin-1-carbonyl)-N1-(1-benzyl-2-(2-hydroxymethyl-pyrrolidin-1-yl)-2-oxo-ethyl-2-pentyl-succinamide (64), N4, N4-methoxymethyl-N1-(1-benzyl-2-(2-hydroxymethyl-pyrrolidin-1-yl)-2-oxo-ethyl-2-pentyl-succinamide (65), N4-hydroxy-N1-(2-oxo-2-(piperidin-1-yl)ethyl)-2-pentyl succinamide (66), 4-((2-(2(hydroxyamino)-2-oxoethyl)heptanamido)-5-oxo-5-(piperidin-1-yl) pentanoic acid (67), N4-hydroxy-N1-(1-piperidine-1-carbonyl)-pentyl)-2-pentyl succinamide (68), N4-hydroxy-N1,N1-(1-diethylamino-1-carbonyl)-2-methyl-propyl)-2-pentyl succinamide (69), N4-hydroxy-N1-(1-piperidine-1-carbonyl)-2-methyl-propyl)-2-pentyl succinamide (70), N4-hydroxy-N1-(1-morpholine-1-carbonyl)-2-methyl-propyl))-2-pentyl succinamide (71), N-hydroxy-3(2-(piperidine-1-carbonyl)pyrrolidine-1-carbonyl)octanamide (72), (5R,8S,12aS)-8-benzyl-5-pentyloctahydropyrrolo[2,1-c][1,4,7] oxadiazacyclundecine-3,6,9(1H)-trione (73), N4-hydroxy-N1-butyl-2-pentylsuccinamide (74), or N-hydroxy-3-(4-isopropyl-2-oxooxazolidine-3-carbonyl)octanamide (75). Pharmaceutical compositions of these compounds are also provided.

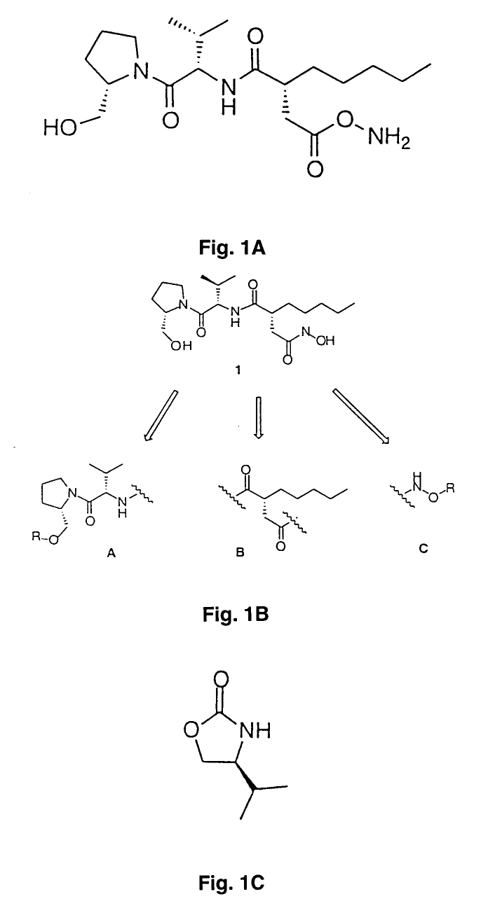

It should be clear that corresponding actinonin analogs or derivatives readily can be formed using the synthetic methods as provided for (S,S,R)-(−)-actinonin (1) (FIG. 1A) and the analogs or derivatives disclosed herein. The synthetic schema detailed herein provide a scalable process for the asymmetric synthesis of (S,R,R)-(−)-actinonin and its analogs and derivatives. The general structure of these compounds can be divided into three fragments. Fragment A is described as a pseudodipeptide. Fragment B is a succinnic acid or other dicarboxylic acid derivative and is composed of a dicarboxylic acid skeleton functionalized with an R³ group at C2 (or at C3 or C4, depending on the acid) or, alternatively, the α- (or β- or γ-) R³ ester. Fragment C is an optionally suitably protected optionally substituted amine. FIG. 1B depicts these fragments for actinonin.

A generalized synthetic scheme to synthesize any of the compounds disclosed herein is shown below. The (−)-actinonin analogs and derivatives are prepared by substituting various amino acids and other reagents to the process. Some substituents would need to be O- or N-protected, e.g., hydroxyl moieties on R⁶ and R⁵ or the amine or a hydroxyl moiety in the R²-amino acid in Fragment A. For example the R2-amino acid may be, but not limited to, phenylalanine, glycine, lysine, valine, or glutamic acid. Fragment B may not be commercially available and would have to be synthesized independently. This involves the use of an Evan's chiral auxiliary 2 (13) (FIG. 1C). This chiral auxiliary route provides an easily prepared and inexpensive alternative to the iron (Fe)-based chiral auxiliary. It is contemplated that other structures may be synthesized using the methods disclosed herein. In the synthetic schema detailed below R¹ is R⁶ in the synthesized structures.

For example, R⁶ may be a pyrrolidine. One set of substituents useful in the method is R⁶ is 2-hydroxymethyl-pyrrolidine, 2-methylpyrrolidine, 2-methylamine-pyrrolidine, methyl-2-pyrrolidine carboxylate, or 2-hydroxycarbamoyl; R² is methyl, benzyl, 4-hydroxybenzyl, methylethyl, 2-methyl propyl, 3-methyl-indolyl; R³ is methyl or pentyl; R⁴ is methylene; and R⁵ is NH₂, OH, NHOH, NHOCH₃, N(CH₃)OH, N(CH₃)OCH₃, NHCH₂CH₃, NH(CH₂CH₃), NHCH₂(2,4-(OCH3)₂Ph, NHCH₂(4-NO₂)Ph, NHN(CH₃)₂, proline, 2-hydroxymethyl pyrrolidine. piperidine or 1-methyl-piperazine. Alternatively, R⁶ may be 2-methyl pyrrolidine, 2-hydroxymethyl pyrrolidine or 2-hydroxycarbamoyl pyrrolidine; R² is methyl, CH₂CH₃, (CH₂)₂CH₃, C(CH₃)₃; R³ is R² or C₄₋₇alkyleneCH₃, R⁴ is methylene; and R⁵ is hydroxylamine. Still again, R⁶ is 2-hydroxymethyl pyrrolidine; R² is methylethyl; R³ is pentyl; R⁴ is methylene; and R⁵ is hydroxylamine such that (S,S,R)-(−)-actinonin is synthesized. In another general example R⁶ may be an optionally substituted or halogenated indoline, indole, pyrrole, or imidazole and R²-R⁵ may be as described herein.

Fragment A:

a) An optionally O-protected R⁶-1-carbonyl-C2-(R²)-methyleneamine from R⁶ and an N-protected R²-amino acid 2,5-dioxo-pyrrolidinyl ester is formed. A suitable agent, e.g. trifluoroacetic acid (TFA) deprotects the N-protected R²-amino.
OH—CO—CH(R²)—NH-boc+OhsuccinNH₂→succinN—O—CO—CH(R²)—NH-boc
succinN—O—CO—CH(R²)—NH-boc+R¹(optionally O-protect)→R¹—CO—CH(R²)—NH2 (A)
Fragment B:

b) Forming an R³-carbonyl-oxazolidone from 4-isopropyl-oxazolidin-2-one and R³-carbonyl chloride;

c) Treating a solution of 4-(S)-isopropyl-oxazolidin-2-one with a solution of a base, e.g., n-butyl lithium in hexanes, and adding an R³-carbonyl chloride to form an R³-carbonyl oxazolidinone.

d) Treating a solution of the R³-carbonyl oxazolidinone sequentially with a base, e.g., lithium diisopropylamide, and with a bromo-R⁴ acid-tert-butyl ester to form an oxazolidine-R³-carbonyl-R⁴-acid tert-butyl ester.

e) Treating a mixture of the an oxazolidine-R³-carbonyl-R⁴-acid tert-butyl ester in tetrahydrofuran and water sequentially with hydrogen peroxide in water and with lithium hydroxide in water to form a C2(R³)—R⁴-dicarboxylic acid tert-butyl ester;

f) Treating a mixture of the C2(R³)—R⁴-dicarboxylic acid 4-tert-butyl ester and hydroxysuccinimide in a solvent, e.g., dioxane or dimethylformamide, with an imide, e.g., dicyclohexylcarbodiimide to form an C2(R³)—R⁴-dicarboxylic acid tert-butyl ester-(2,5-dioxo-pyrrolidin-1-yl) ester.
Cl—CO—R³+4-isopropropyl-oxazolidin-2-one→R³—CO-oxazolidone
R³—CO-oxazolidone+t-buBrR⁴ ester→oxaz-CO—CH(R³)—CH(R⁴—COO-tbu
oxaz-CO—CH(R³)—R⁴—COO-tbu→HO—CO—CH(R³)—R⁴—COO-tbu
HO—CO—CH(R³)—R⁴—COO-tbu+OHsuccinNH→succinN—OCOCH(R³)—R⁴—COO-tbu (B)
Fragment A+Fragment B:

g) Treating a solution of the optionally O-protected R⁶-1-carbonyl-2-(R²)-methyleneamine in a solvent, e.g., tetrahydrofuran, sequentially with triethylamine and with the C2(R³)—R⁴-dicarboxylic acid tert-butyl ester-(2,5-dioxo-pyrrolidin-1-yl) ester to form an optionally O-protected R¹-1-carbonyl-2-(R²)-carbamoyl-methylene(R³)—R⁴-carboxylic acid tert-butyl ester.

h) Treating a solution of the optionally O-protected R⁶-1-carbonyl-C2(R²)-carbamoyl-methylene(R³)—R⁴-carboxylic acid tert-butyl ester in a solvent, e.g., methylene chloride, with TFA to form an optionally O-protected R⁶-1-carbonyl-C2(R²)-carbamoyl-methylene(R³)—R⁴-carboxylic acid.

i) Treating the optionally O-protected R⁶-1-carbonyl-2C—(R²)-carbamoyl-methylene(R³)—R⁴-carboxylic acid and hydroxysuccinamide with an imide, e.g., DCC, to form an optionally O-protected R⁶-1-carbonyl-C2(R²)-carbamoyl-methylene(R³)—R⁴-carboxylic acid 2,5-dioxo-pyrrolidin-1-yl ester.
succinN—OCOCH(R³)—R⁴—COO-tbu+R¹—CO—CH(R²)—NH2→R¹COCH(R²)NHCOCH(R³)R⁴—COO-tbu→R¹COCH(R²)NHCOCH(R³)R⁴—COOH
R¹COCH(R²)NHCOCH(R³)R⁴—COOH+OHsuccinNH2→R¹COCH(R²)NHCOCH(R³)R⁴—COO—Nsuccin (A—B)
Fragment C+Fragment A—B:

j) Treating a suspension of an optionally O-protected R⁵ or its chloride in a solvent, e.g., DMF, sequentially with triethylamine and with a solution of the O-protected R⁶-1-carbonyl-C2(R²)-carbamoyl-methylene(R³)—R⁴-carboxylic acid 2,5-dioxo-pyrrolidin-1-yl ester in a solvent, e.g., DMF, to form an R⁶-1-carbonyl-C2(R²)-carbamoyl-methylene(R³)—R⁴-carbonyl-R⁵ where R⁶ and R⁵ may be independently O-protected.

k) Hydrogenating R⁶ and R⁵, if R⁶ and R⁵ independently have an O-protecting group, with hydrogen gas and a catalyst, e.g., palladium hydroxide in activated carbon, to form (S,S,R)-(−)-actinonin or its derivative or analog.
R¹COCH(R²)NHCOCH(R³)R⁴—COO—Nsuccin+R⁵(optionally O-protect)→R¹COCH(R²)NHCOCH(R³)R⁴—CO—R⁵ (A—B—C)

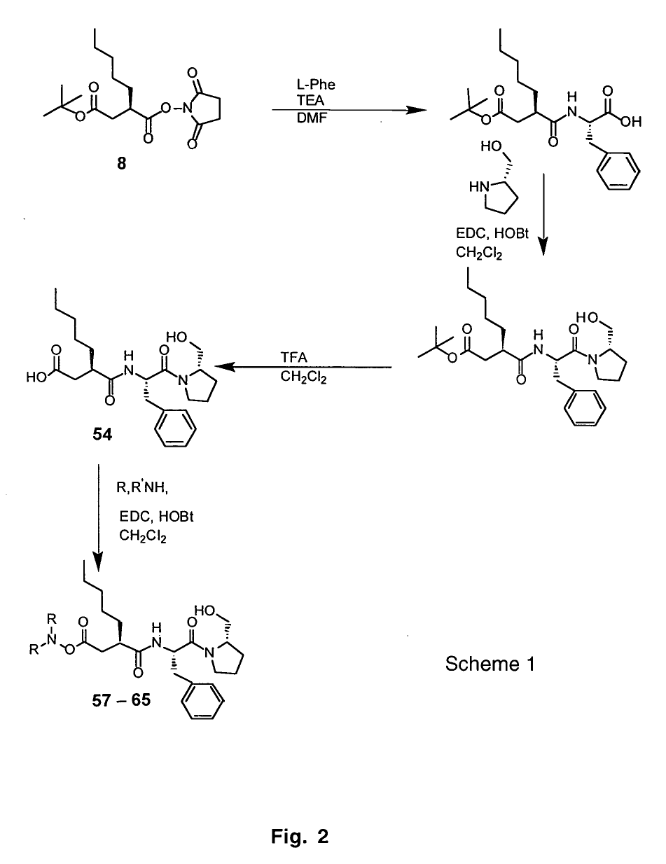

An alternative synthetic scheme can be used for those analogs or derivatives of actinonin where R² is benzyl, R⁵ is a substituted amide, e.g., NHOCH₃, N(CH₃)OCH₃, NHCH₂CH₃, NH(CH₂CH₃)₂, NHCH₂(2,4-(OCH3)₂Ph, NHCH₂(4-NO₂)Ph, NHN(CH₃)₂, piperidine or 1-methyl-piperazine and R⁶ is 2-hydroxymethyl-pyrrolidine. In this instance L-phenylalanine is added to fragment B followed by 2-hydroxymethyl pyrrolidine. The t-butyl ester of Fragment B is removed and R⁵ is added to form the compounds. Scheme 1 details the synthesis using structural formulas (FIG. 2).

Fragment B+L-phenylalanine:

a) Treating a solution of L-phenylalanine in a solvent, e.g., DMF, sequentially with triethylamine and with the 2-pentylsuccinic acid 4-tert-butyl ester 4-(2,5-dioxo-pyrrolidin-1-yl) ester to form an 3-(1-Carboxy-2-phenyl-ethylcarbamoyl)-octanoic acid tert-butyl ester.
succinN-OCOCH((CH2)₄CH₃)—CH₂—COO-tbu+L-phe→HO—CO—CH(CH₂(C₆H₅))—NHCO—CH((CH2)₄CH₃)—CH₂—COO-tbu→
Adding R⁶ or 2-hydroxymethyl pyrrolidine:

b) Coupling 2-hydroxymethyl pyrrolidine to 3-(1-carboxy-2-phenyl-ethylcarbamoyl)-octanoic acid tert-butyl ester in a solvent, e.g., methylene chloride, and in the presence of EDC and HOBT to form 3-[1-(2-hydroxymethyl-pyrrolidin-1-yl)-2-benzylcarbamoyl]-octanoic acid 4-tert-butyl ester.

c) Treating a solution of 3-[1-(2-hydroxymethyl-pyrrolidin-1-yl)-2-benzylcarbamoyl]-octanoic acid 4-tert-butyl ester in a solvent, e.g., methylene chloride, with TFA to form 3-[1-(2-hydroxymethyl-pyrrolidin-1-yl)-2-benzylcarbamoyl]-octanoic acid.
HOCOCH(CH₂(C₆H₅))—NHCO—CH((CH2)₄CH₃)—CH₂—COO-tbu+HOCH3-pyrrolidine+cleave tbu→HOCH₂—Npyr-COCH(CH₂(C₆H₅))—NHCO—CH((CH2)₄CH₃)—CH₂—COOH
Adding R⁵:

d) Treating a suspension of R⁵ in a solvent, e.g., methylene chloride, and in the presence of EDC and HOBT with a solution of 3-[1-(2-hydroxymethyl-pyrrolidin-1-yl)-2-benzyl carbamoyl]-octanoic acid in methylene chloride to form N4(R⁵)—N1-[1-benzyl-2(2-hydroxymethyl-pyrrolidin-1-yl)-2-oxo-ethyl]-2-pentyl-succinamide.
HOCH₂NpyrCOCH(CH₂(C₆H₅))—NHCO—CH((CH2)₄CH₃)—CH₂—COOH+R⁵→HOCH₂NpyrCOCH(CH₂(C₆H₅))—NHCO—CH((CH2)₄CH₃)—CH₂—CO—R⁵

It is also contemplated that these analogs and derivatives are to be used as anti-tumor agents or for the use in the treatment of neoplastic diseases or to inhibit growth of tumor cells comprising the neoplastic disease by the methods disclosed herein. It is contemplated that contacting a neoplastic or tumor cell will inhibit the growth thereof. Contact may be effected by any means standard in the art. The neoplastic cell or tumor cell may be contacted in vitro, in vivo or ex vivo. The neoplastic disease or neoplastic or tumor cells comprising the same, may be, but not limited to, a human ovarian carcinoma, a prostate carcinoma, a mammary carcinoma, a head and neck squamous cell carcinoma, a non-small-cell-lung-cancer adenocarcinoma, non-small-cell-lung-cancer squamous cell carcinoma, or acute myologenous leukemia.

It is specifically contemplated that pharmaceutical compositions may be prepared using the novel actinonin analogs and derivatives of the present invention. In such a case, the pharmaceutical composition comprises the novel compounds of the present invention and a pharmaceutically acceptable carrier. When used in vivo for therapy, the compounds of the present invention are administered to the patient or an animal in therapeutically effective amounts, i.e., amounts that eliminate or reduce the tumor burden. A person having ordinary skill in this art would readily be able to determine, without undue experimentation, the appropriate amounts, as individual doses or as an overall dosage and routes of administration of the actinonin compounds analogs and derivatives of the present invention. Such determination is made based on, inter alia, the state of the art, the neoplastic disease and the patient history of the individual in need of treatment for the neoplastic disease.

The following examples are given for the purpose of illustrating various embodiments of the invention and are not meant to limit the present invention in any fashion.

### EXAMPLE 1

### General Methods and Materials

The ¹H NMR and ¹³C NMR spectra were recorded on a Bruker 400 MHz using tetramethylsilane as the internal standard. High resolution mass spectra were obtained at the Mass Spectrometry Facility, University of California at Riverside. All reagents are obtained either from Sigma-Aldrich® or from Lancaster® and vacuum dried under P₂O₅ overnight before use. All solvents were reagent grade and distilled before use. Silica gel used for chromatography, MN-Kieselgel 60, was purchased from Brinkman Instruments Inc. All reactions were carried out under argon using glassware dried in an oven at 80° C. overnight and cooled under vacuum. The reaction mixtures were mechanically stirred using a magnetic stirring bar and stirring plate. Melting points were determined using a Mel-Temp II melting point apparatus fitted with a digital Barnart 115 thermocouple thermometer, and are uncorrected.

### EXAMPLE 2

### General Synthetic Protocol for Actinonin and Specific Analogs

For actinonin and those analogs or derivatives where R⁶ is 2-methyl pyrrolidine, 2-hydroxymethyl pyrrolidine or 2-hydroxycarbamoyl pyrrolidine; and R² is methyl, CH₂CH₃, (CH₂)₂CH₃, C(CH₃)₃ and R³ is R² or C₄₋₇alkyleneCH₃, the following protocol can be used:

a) Coupling of a suitably O-protected methoxypyrrolidine or a derivative thereof with a suitably N-protected amino acid 2,5-dioxo-pyrrolidinyl ester to form suitable N,O-protected methylpyrrolidine-1-carbonyl-2-methylamine.

b) Deprotection of the Nprotecting group with a suitable deprotecting agent such as trifluoracetic acid to yield the corresponding pyrrolidine-1-carbonyl-2-methylamine or a derivative thereof.

c) Treatment of a solution of a chiral auxiliary, such as 4-(S)-isopropyl-oxazolidin-2-one 2, with a solution of a suitable base, such as n-BuLi in hexanes, followed by the addition of an alkynoyl chloride to yield an alkynoyloxazolidinone.

d) Treatment of a solution of the alkynoyloxazolidinone with lithium diisopropylamide, or similar bases, followed by bromoacetic acid tert-butyl ester to yield an oxazolidine-carbonyl-alkynoic acid tert-butyl ester.

e) Treatment of a mixture of an oxazolidine-carbonyl-alkynoic acid tert-butyl ester in THF/water with hydrogen peroxide in water followed by lithium hydroxide in water to yield an alkylsuccinic acid 4-tert-butyl ester.

f) Treatment of a mixture of an alkylsuccinic acid 4-tert-butyl ester and hydroxysuccinimide 7 in a suitable solvent, such as dioxane or dimethylformamide, with dicyclohexylcarbodiimide, or similar imides, to afford an alkylsuccinic acid 4-tert-butyl ester 4-(2,5-dioxo-pyrrolidin-1-yl) ester.

g) Treatment of a solution of a pyrrolidine-1-carbonyl-2-methylamine or a derivative thereof in a suitable solvent is treated with triethylamine followed by an alkylsuccinic acid 4-tert-butyl ester 4-(2,5-dioxo-pyrrolidin-1-yl) ester to yield a pyrrolidine-1-carbonyl-2-methylalkyl-carbamoyl-alkynoic acid tert-butyl ester or a derivative thereof.

h) Treatment of a pyrrolidine-1-carbonyl-2-methylalkyl-carbamoyl-alkynoic acid tert-butyl ester or a derivative thereof in a suitable solvent with trifluoroacetic acid to yield a pyrrolidine-1-carbonyl-2-methyl-alkylcarbamoyl-alkynoic acid or a derivative thereof.

i) Treatment of a pyrrolidine-1-carbonyl-2-methyl-alkylcarbamoyl-alkynoic acid or a derivative thereof and hydroxysuccinamide (7) with dicyclohexylcarbodiimide or any suitable imide to afford a pyrrolidine-1-carbonyl-2-methylalkylcarbamoyl)-alkynoic acid 2,5-dioxo-pyrrolidin-1-yl ester or a derivative thereof.

j) Treatment of a suspension of O-benzylhydroxyamine hydrochloride 18 in a suitable solvent with triethylamine followed by a solution of a pyrrolidine-1-carbonyl-2-methylalkylcarbamoyl)-alkyoic acid 2,5-dioxo-pyrrolidin-1-yl ester or a derivative thereof in a suitable solvent to afford N4-benzyloxy-N1-(1-(pyrrolidine-1-carbonyl)-2-methylalkyl-2-alkyl-succinamide or a derivative thereof.

k) Hydrogenation of a N-benzyloxy-N1-(1-pyrrolidine-1-carbonyl)-2-methylalkyl-2-alkyl-succinamide or a derivative thereof with hydrogen gas and a suitable catalyst wherein actinonin or its analogs are thereby formed.

### EXAMPLE 3

### Specific Synthesis of (S,R,R)-(−)-actinonin

(S,R,R)-(−)-Actinonin is divided into three fragments A, B and C as previously disclosed. Fragment A, described as a pseudodipeptide, is composed of a (S)-prolinol and a (S)-valine. Fragment B, described as a succinnic acid derivative, is composed of a succinnic acid skeleton functionalized with an n-pentyl group at C2 or, alternatively, an α-pentylsuccinate. Fragment C is a suitably protected hydroxylamine (FIG. 1B).

Synthesis of Fragment A

Fragment A is prepared by coupling a suitably N-protected and carboxy-activated (S)-valine and a suitably O-protected (S)-prolinol. The α-pentylsuccinate fragment B is synthesized using an Evan's chiral auxiliary 2 (13) (FIG. 1C).

Synthesis of Fragment B

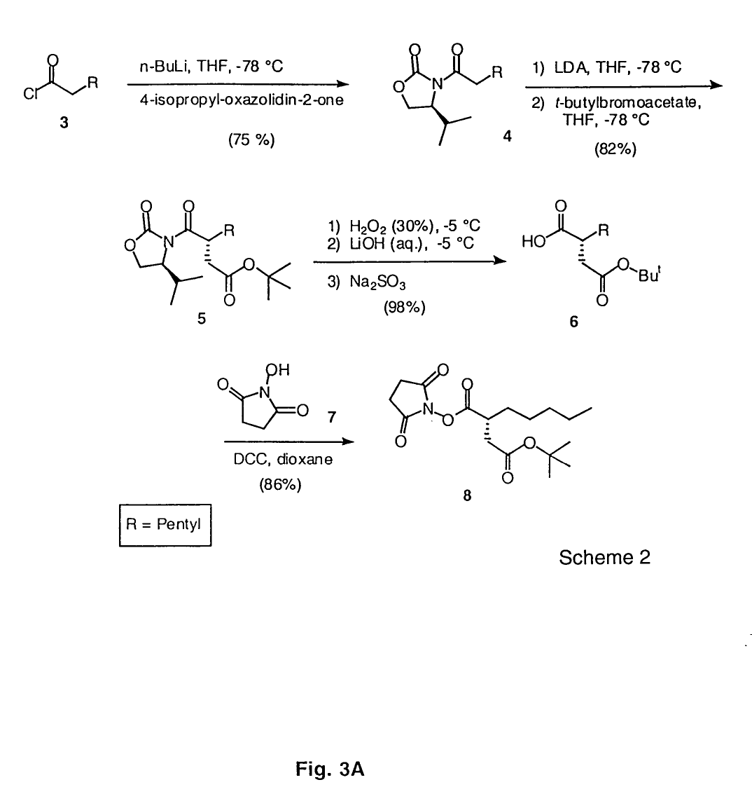

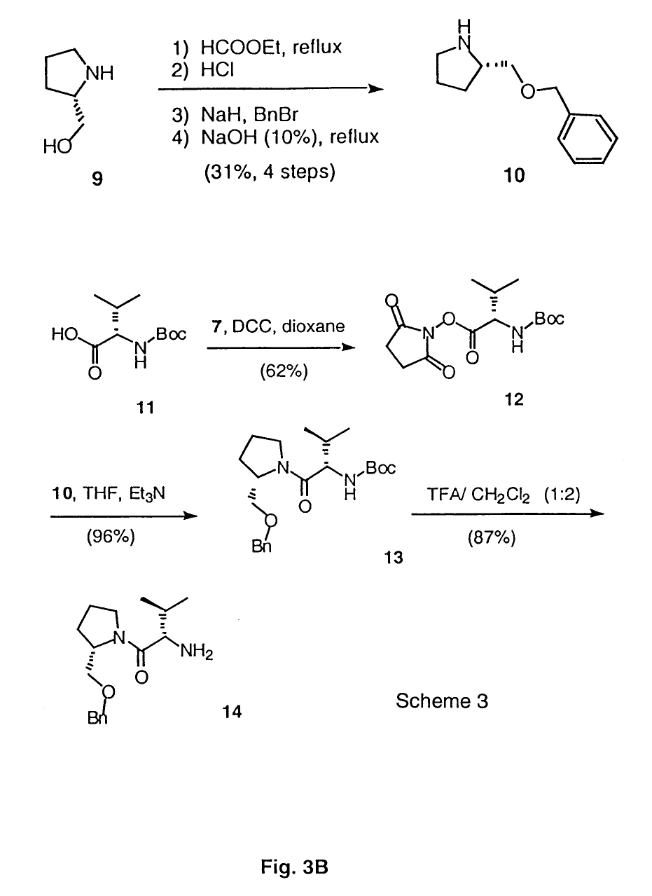

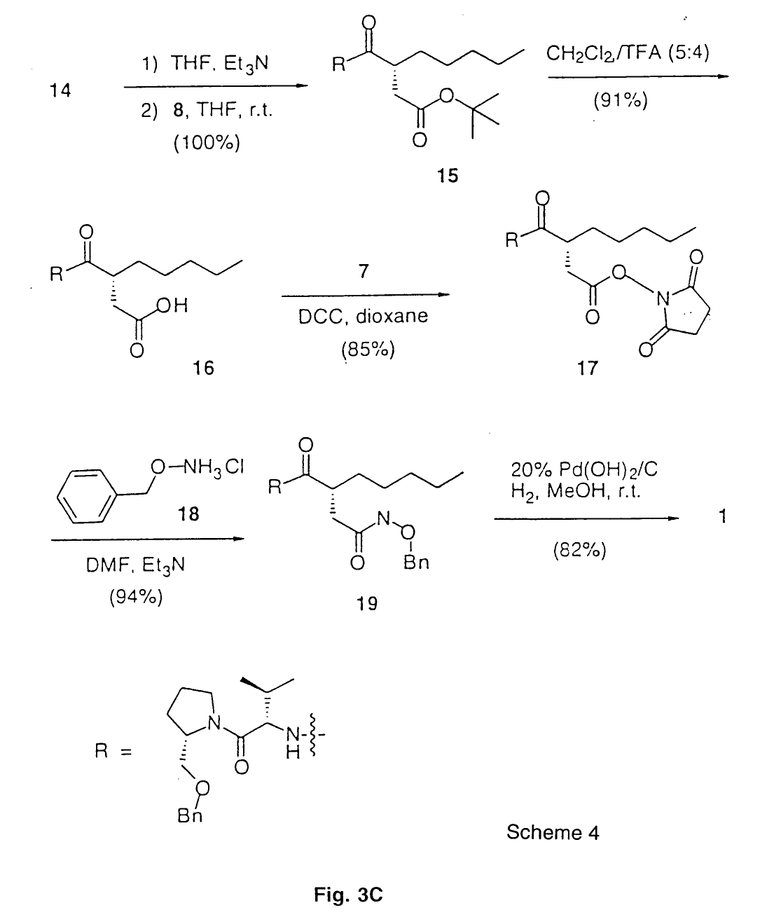

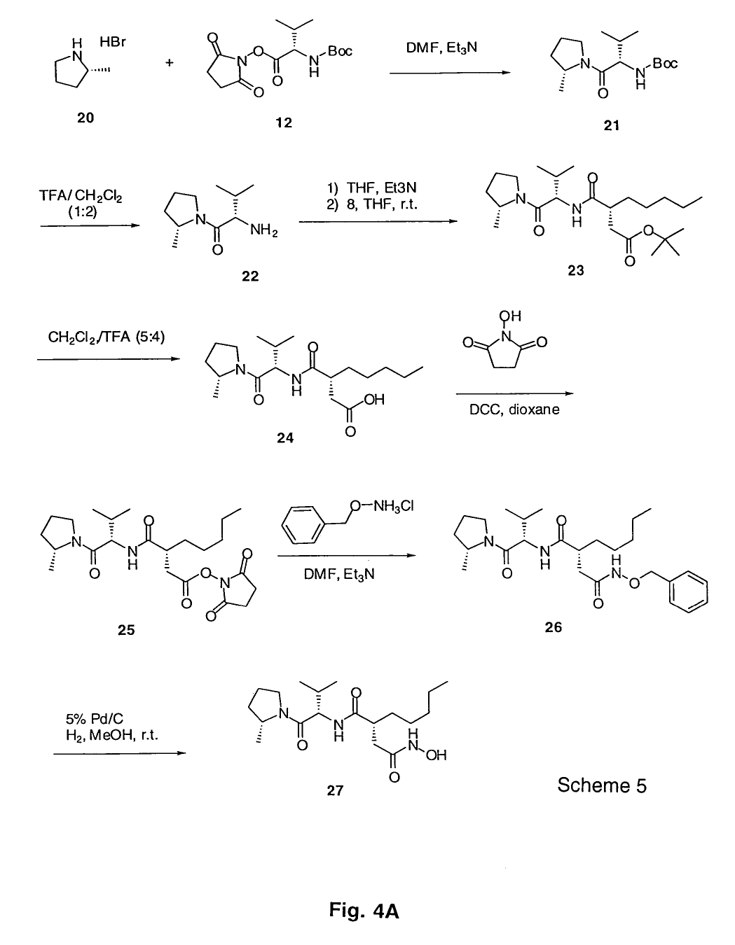

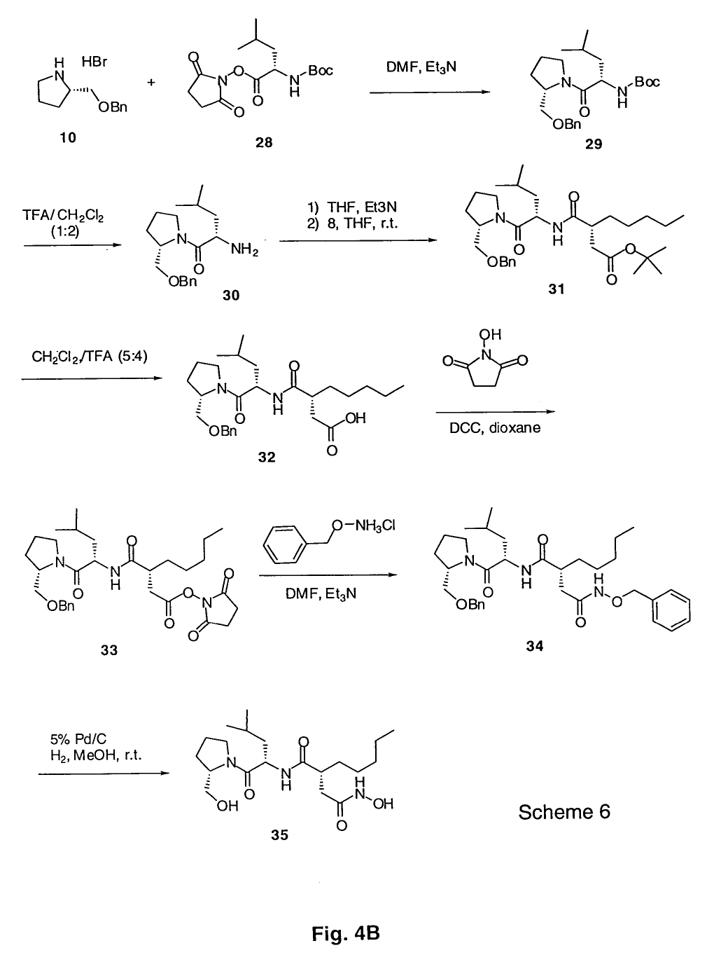

The synthesis of Fragment B is comprised of the following steps: (a) a solution of 4-(S)-isopropyl-oxazolidin-2-one 2 in THF at a temperature of −78° C. is treated with a solution of n-BuLi in hexanes, or any suitable base; (b) heptanoyl chloride 3 is added to the mixture to yield 3-heptanoyl-4-(S)-isopropyl-oxazolidin-2-one 4; (c) treatment of a solution of 4 in THF with lithium diisopropylamide, or similar bases, followed by bromoacetic acid tert-butyl ester yield 3-(4-(S)-isopropyl-2-oxo-oxazolidine-3-(S)-carbonyl)octanoic acid tert-butyl ester 5; (d) treatment of a mixture of 5 in THF/water with hydrogen peroxide in water followed by lithium hydroxide in water at 0° C. to yield 2-(R)-pentylsuccinic acid 4-tert-butyl ester 6; (e) treatment of a mixture of 6 and hydroxysuccinimide 7 in dimethylformamide or any suitable solvent, such as dioxane, with dicyclohexylcarbodiimide, or similar imides, to afford 2-(R)-pentylsuccinic acid 4-tert-butyl ester (should this be 1, if it is 4-t-butyl ester?)4-(2,5-dioxo-pyrrolidin-1-yl) ester 8 (Scheme 2, FIG. 3A).

Synthesis of Fragment A

The synthesis is comprised of the following steps: (a) a solution of 2-(S)-benzyloxymethylpyrrolidine 10 in THF is treated with triethylamine followed by a solution of 2-tert-butoxycarbonylamino-3-methylbutyric acid 2,5-dioxo-pyrrolidin-1-yl 12 in THF to yield (1-(2-benzyloxymethyl-pyrrolidine-1-carbonyl)-2-methyl-propyl)-carbamic acid tert-butyl ester 13; which is then (b) dissolved in methylene chloride, or any suitable solvent, and treated with trifluoroacetic acid or any suitable Boc-deprotecting agent to yield 2-amino-1-(2-benzyloxymethylpyrrolidin-1-yl)-3-methylbutan-1-one 14 (Scheme 3, FIG. 3B).

A problem can occur with overhydrogenation involving the hydroxylamine group being converted to the corresponding amide. However, this could be avoided by carefully monitoring the amount of hydrogen consumed during hydrogenation. In some occassions, when the overreduction product is observed in TLC, the reaction is quenched, the product is isolated, and the starting material resubjected to hydrogenation.

Synthesis of (−)-actinonin Using Fragments A, B and C

The coupling of fragment A and B is comprised of the following steps: (a) a solution of 14 in dimethylformide or any suitable solvent is treated with triethylamine followed by a solution of 8 in dimethylformamide or any suitable solvent to yield 3-(1-(2-(S)-benzyloxymethylpyrrolidine-1-carbonyl)-2-(S)-methylpropyl-carbamoyl)-octanoic acid tert-butyl ester 15; (b) treatment of 15 in dichloromethane with trifluoroacetic acid to yield 3-(1-(2-benzyloxymethyl-pyrrolidine-1-carbonyl)-2-methyl-propyl carbamoyl)-octanoic acid 16; (c) treatment of a solution 16 and hydroxysuccinamide 7 with dicyclohexylcarbodiimide or any suitable imide to afford 3-(1-(2-benzyloxymethyl-pyrrolidine-1-carbonyl)-2-methylpropylcarbamoyl)-octanoic acid 2,5-dioxo-pyrrolidin-1-yl ester 17; (d) fragment C is introduced by treatment of a suspension of O-benzylhydroxyamine hydrochloride 18 in dimethylformamide with triethylamine followed by a solution of 17 in dimethylformamide to afford N4-benzyloxy-N1-(1-(2-benzyloxymethyl-pyrrolidine-1-carbonyl)-2-methyl-propyl)-2-pentyl-succinamide 19. (f) hydrogenating 19 with hydrogen gas and palladium hydroxide in activated carbon wherein actinonin 1 is thereby formed (Scheme 4, FIG. 3C).

### EXAMPLE 4

### Synthesis of Specific Compounds in Schemes 1, 2 and 3

### Synthesis of 3-heptanoyl-4-(S)-isopropyl-oxazolidin-2-one (4)

To a solution of oxazolidone (2) (30.2 g, 0.234 mol) in 400 mL THF at −78° C. was added n-BuLi (2.5 M in hexanes, 96.0 mL, 0.240 mol) dropwise over a period of 30 min. The solution was stirred at −78° C. for an hour. Heptanoyl chloride (37.15 g, 0.250 mol) was gradually added by syringe and the resulting solution stirred at −78° C. for 1 h and then warmed to 0° C. A saturated aqueous solution of NH₄Cl (100 mL) was added and the mixture gradually warmed to room temperature. The mixture was extracted with ethyl acetate (3×20 mL) and the combined organic layers were washed with brine and dried over anhydrous MgSO₄. The solvent was removed by a rotary evaporation and the yellowish residue chromatographed on a silica gel column using Hexane/EtOAc (8:1) as the eluant to give 4 as a colorless oil (46.272 g, 0.192 mol, 82%). ¹H NMR (400 MHz, CDCl₃) δ 0.87 (t, J=7.2 Hz, 3 H), 0.88 (d, J=16.9 Hz, 3 H), 0.90 (d, J=16.9 Hz, 3 H), 1.24-1.39 (m, 6 H), 1.59-1.69 (m, 2 H) 2.31-2.43 (m, 1 H), 2.81-3.02 (m, 2 H), 4.19-4.29 (m, 2 H), 4.41-4.45 (m, 1 H); ¹³C NMR (100 MHz, CDCl₃) δ 13.9, 14.5, 17.9, 22.4, 24.3, 28.3, 28.7, 31.4, 35.4, 58.3, 63.2, 154.0, 173.4.

### Synthesis of 3-(S)-(4-(S)-isopropyl-2-oxo-oxazolidine-3-carbonyl)-octanoic acid tert-butyl ester (5)

To a solution of 4 (37.082 g, 0.154 mol) in THF (300 mL) at −78° C. is added LiHMDS in THF (1.0 M, 0.162 mol, 162 mL) via cannula. The solution is stirred at −78° C. for 1 h. tert-Butylbromoacetate is added and then stirred for 30 more minutes. It is then gradually warmed to 0° C. and stirred at this temperature for 30 min. To this solution is gradually added 100 mL saturated aqueous solution of NH₄Cl. The bulk of the THF is removed by rotary evaporation and the remaining mixture was extracted with CH₂Cl₂ (100 mL×3). The combined CH₂Cl₂ extracts is washed consecutively with 1N NaOH, 1 N HCl, saturated NaHCO₃, and brine. It is then dried over MgSO₄ and concentrated in vacuo. The residue is chromatographed in silica gel using hexane/ethyl acetate (5:1) as eluant to afford 5. ¹H NMR (400 MHz, CDCl₃) δ 0.85 (t, J=6.8 Hz, 3 H), 0.91 (d, J=8.15 Hz, 3 H), 0.93 (d, J=8.15 Hz, 3 H), 1.18-1.35 (m, 6 H), 1.40 (s, 9 H), 1.35-1.49 (m, 1 H), 1.56-1.66 (m, 1 H), 2.31-2.39 (m, 1 H), 2.44 (dd, J=16.6, 4.5, 1 H), 2.73 (dd, J=16.6, 10.3, 1 H), 4.11-4.20 (m, 1 H), 4.44 (m, 1 H); ¹³C NMR (75.45 MHz, CDCl₃) δ 13.9, 14.4, 17.9, 22.4, 26.4, 27.9, 28.0, 31.4, 31.6, 31.7, 37.0, 39.2, 58.6, 62.9, 80.3, 153.5, 171.2, 175.8. HRMS calcd for C₁₉H₃₄NO₅ (MH⁺): 356.2436. Found (DCI): 356.2418 (Δ=5.3 ppm).

### Synthesis of 2-(R)-pentylsuccinic acid 4-tert-butyl ester (6)

A solution of 5 (25.5 g, 71.73 mmol) in THF (300 mL) and water (75 mL) under Ar is cooled to 0° C. To this is added via syringe H₂O₂ (30% in water, 30.6 mL, 0.300 mol) gradually over a period of 15 minutes. LiOH.H₂O (4.92 g, 0.12 mol) in water (150 mL) is added via syringe. The mixture is stirred for 3 h after which the septum is removed and Na₂SO₃ (37.8 g, 0.300 mol) in water (225 mL) is added slowly. The bulk of the THF is removed by rotavap at a bath temperature between 25 -30° C. The residue is extracted with CH₂Cl₂ (100 ml×3). The combined organic layer is washed consecutively with 0.5 N HCl (100 mL), sat. NaHCO₃ (100 mL), water (100 mL) and brine (100 mL). The solution is then dried over Na₂SO₄ and concentrated in vacuo. The residue is recrystallized in hexane/ethyl acetate to afford the Evan's chiral auxiliary 2 as white needles. The aqueous layer from the first extraction was acidified to pH 1 and extracted with ethyl acetate (100 mL×3). The combined organic layer is washed with 0.5 N HCl (100 mL) and then brine (100 mL). It is then dried over Na₂SO₄ and concentrated to yield 6 as a colorless oil (17.198 g, 70.39 mmol, 98%). ¹H NMR (400 MHz, CDCl₃) δ 0.88 (t, J=6.8 Hz, 3 H), 1.21-1.37 (m, 6 H), 1.43 (s, 9 H), 1.40-1.70 (m, 2 H), 2.61 (dd, J=9.3 Hz, 1 H), 2.38 (dd, J=16.4, 5.1, 1 H), 2.61 (dd, J=16.4, 9.3, 1 H), 2.78 (m, 1 H); ¹³C NMR (100 MHz, CDCl₃) δ 13.9, 17.5, 17.9, 22.3, 26.5, 27.9, 31.5, 37.0, 41.3, 94.9, 80.0, 171.1, 181.1. HRMS calcd for C₁₃H₂₅O₄ (MH⁺): 245.1752. Found (DCI): 245.1766 (Δ=5.2 ppm).

### Synthesis of 2-(R)-pentyl-succinic acid 4-tert-butyl ester 4-(2,5-dioxo-pyrrolidin-1-yl) ester (8)

To a solution of 6 (7.149 g, 29.26 mmol) and N-hydroxysuccinimide (6.90 g, 60.0 mmol) in 1,4-dioxane (40 mL) at r.t. is added via cannula a solution of DCC (8.25 g, 40 mmol) in 1,4-dioxane (20 mL). The mixture is stirred overnight, afterwhich it is filtered through celite and the solids washed with acetone. The filtrate is concentrated in vacuo and the residue redissolved in acetone. The acetone solution is cooled to 0° C. and filtered again. The filtrate is concetrated in vacuo and dissolved in ethyl acetate/THF. The solution is washed with sat. solution of NaHCO₃ (100 mL) and then brine (100 mL). It is then dried over Na₂SO₄ and concentrated to yield 8 as a white solid (8.93 g, 25.16 mmol, 86%). ¹H NMR (400 MHz, CDCl₃) δ 0.85 (t, J=6.1 Hz, 3 H), 1.27-1.38 (m, 4 H), 1.43 (s, 9 H), 1.59-1.67 (m, 2 H), 1.71-1.78 (m, 2 H), 2.47 (dd, J=16.7, 8.0 Hz, 1 H), 2.72 (dd, J=16.7, 8.0, 1 H), 2.81 (bs, 4 H), 3.07-3.14 (m, 1 H); ¹³C NMR (100 MHz, CDCl₃) δ 14.3, 19.4, 22.7, 25.9, 26.5, 28.3, 31.8, 32.1, 37.3, 39.5, 81.8, 169.3, 170.3, 170.8. HRMS calcd for C₁₇H₃₁N₂O₆ (MNH₄⁺): 359.2181. Found (DCI): 359.2167 (Δ=4.2 ppm).

### Synthesis of 2-(S)-benzyloxymethyl-pyrrolidine (10)

A solution of 2-S-methanol-pyrrolidine (10.0 g, 99.0 mmol) in ethylformate (50 mL) is refluxed for 3 h. It is then cooled and concentrated in vacuo to afford a yellowish oil which is taken in ethyl acetate (50 mL) and washed successively with 1N HCl (50 mL×3), sat. NaHCO₃ (50 mL), and brine (50 mL). The solution is dried over Na₂SO₄, concentrated in vacuo, and dried further overnight under vacuum. The yellowish oil is dissolved in THF (100 mL), cooled to 0° C., and treated with NaH (6.0 g, 0.250 mol). Benzyl bromide (34.2 g, 0.20 mol) is added dropwise. The mixture is stirred overnight after which it is cooled to 0° C. and a saturated solution of NH₄Cl is added slowly. The layers are separated and the THF layer is diluted with ethyl acetate (50 mL). This solution is washed with 1 N HCl (50 mL) and then brine (50 mL). The solution is then concentrated in vacuo. The residue is mixed with 10% NaOH (100 mL) and refluxed overnight. The mixture is then extracted with ethyl acetate (50 mL×3). The organic layers are combined and washed successively with 50 mL portions of 1 N HCl, sat. NaHCO₃, water, and brine. It is then dried over Na₂SO₄ and concentrated. The residue is chromatographed in silica gel using ethyl acetate as eluant to yield 10 as a yellowish oil (5.84 g, 30.6 mmol, 31%). ¹H NMR (400 MHz, CDCl₃) δ 1.33-1.38 (m, 1H), 1.63-1.77 (m, 3 H), 2.75-2.81 (m, 1H), 2.87-2.91 (m, 1 H), 3.22-3.46 (m, 3 H), 4.43-4.50 (m, 2 H) 7.20-7.28 (m, 5 H); ¹³C NMR (100 MHz, CDCl₃) δ 4.7, 27.4, 33.6, 45.9, 57.4, 72.6, 73.3, 127.0, 127.1, 127.6, 127.8, 137.9.

### Synthesis of (1-(2-benzyloxymethyl-pyrrolidine-1-carbonyl)-2-methyl-propyl)-carbamic acid tert-butyl ester (13)

To a solution of 10 (2.67 g, 14.0 mmol) and triethylamine (5 mL) in THF (10 mL) at r.t. is added a solution of 12 (4.40 g, 14.0 mmol) via cannula. After complete addition, the solution is stirred overnight. THF is removed in vacuo and the residue taken in ether. The ether solution is washed successively with 50 mL portions of 0.5 N HCl, sat. NaHCO₃, water, and brine. It is then dried over Na₂SO₄ and concentrated. The residue is chromatographed in silica gel using hexane/ethyl acetate (3:1:) as eluant to yield 13 as a colorless oil (4.75 g, 12.16 mmol, 87%). %). ¹H NMR (400 MHz, CDCl₃) δ 0.88 (t, J=6.7 Hz, 3 H), 0.94 (d, J=6.7, 3 H), 1.43 (s, 9 H), 1.84-2.09 (m, 5 H), 2.00-2.10 (m, 1 H); 2.28-2.36 (m 1 H), 2.49 (dd, J=16.7, 6.2 Hz, 1 H), 2.74 (dd, J=16.7, 8.2, 1 H), 3.47-3.52 (m, 1 H), 3.55-3.60 (m, 2 H), 3.62-3.68 (m, 2 H), 4.25-4.29 (m, 1 H), 4.32-4.35 (m, 1 H), 4.48-4.52 (m, 2 H), 5.26-5.31 (m, 1 H), 7.25-7.36 (m, 5 H); ¹³C NMR (100 MHz, CDCl₃) δ 17.8, 19.8, 24.8, 27.6, 28.7, 31.9, 48.0, 57.0, 57.3, 70.4, 73.5, 79.7, 127.3, 127.5, 128.2, 138.9, 157.1, 172.5. HRMS calcd for C₂₂H₃₄N₂O₄ (MH⁺): 391.2596. Found (DCI): 391.2612 (Δ=−3.9 ppm).

### Synthesis of 2-amino-1-(2-benzyloxymethyl-pyrrolidin-1-yl)-3-methyl-butan-1-one (14)

To a solution of 13 (4.75 g, 12.16 mmol) in CH₂Cl₂ (10 mL) is added TFA (10 mL). The solution is stirred for 1 h and then quenched with a saturated solution of NaHCO₃ (50 mL). The mixture is extracted with CH₂Cl₂ (50 mL×2). The combined organic layers are washed with 50 mL portions of sat. NaHCO₃, water, and brine. It is then dried over Na₂SO₄ and concentrated to give pure 14 as a yellow oil (3.07 g, 10.56 mmol, 87%). ¹H NMR (400 MHz, CDCl₃) δ 0.87 (t, J=6.8 Hz, 3 H), 0.91 (d, J=6.8, 3 H), 1.65-1.79 (m, 2 H), 1.79-1.96 (m, 2 H), 1.99-2.17 (m, 1 H); 3.27-3.45 (m 1 H), 3.46-3.54 (m, 1 H), 3.59-3.62 (m, 1 H), 4.33-4.38 (m, 1 H), 4.46-4.54 (m, 2 H), 7.27-7.42 (m, 5 H); ¹³C NMR (100 MHz, CDCl₃) δ 16.9, 19.8, 24.4, 27.2, 47.2, 56.6, 58.3, 70.1, 73.1, 127.3, 127.4, 128.2, 138.4, 173.8. HRMS calcd for C₁₇H₂₇N₂O₂ (MH⁺): 291.2072. Found (DCI): 291.2088 (Δ=−5.3 ppm).

### Synthesis of 3-(R)-(1-(2-(S)-benzyloxymethyl-pyrrolidine-1-carbonyl)-2-(S)-methyl-propylcarbamoyl)-octanoic acid tert-butyl ester (15)

To solution of 14 (3.06 g, 10.56 mmol), triethylamine (5 mL), and THF (100 mL) is added a solution of 8 in THF (40 mL). The solution is stirred overnight and then washed with 50 mL portions of 1 N HCl, sat. NaHCO₃, water, and brine. It is then dried over Na₂SO₄ and concentrated in vacuo. The residue is chromatographed in silica gel using hexane/ethyl acetate (3:1) as eluant to give 15 as a clear colorless oil (5.25 g, 10.17 mmol, 96%). ¹H NMR (400 MHz, CDCl₃) δ 0.85 (t, J=6.3 Hz, 3 H), 0.92 (d, J=13.1, 3 H), 0.94 (d, J=13.1, 3 H), 1.09-1.34 (m, 6 H), 1.43 (s, 9 H), 1.58-1.66 (m, 2 H), 1.68-1.76 (m, 2 H), 1.85-1.97 (m, 4 H), 2.00-2.10 (m, 1 H); 2.28-2.36 (m 1 H), 2.49 (dd, J=16.7, 6.2 Hz, 1 H), 2.74 (dd, J=16.7, 8.2, 1 H), 3.10-3.21 (m, 2 H), 3.46-3.52 (m, 2 H), 3.67-3.77 (m, 2 H), 4.28-4.33 (m, 2 H), 4.49 (s, 2 H), 4.59-4.63 (m, 2 H), 7.29-7.37 (m, 5 H); ¹³C NMR (100 MHz, CDCl₃) δ 13.8, 17.4, 19.0, 22.3, 24.3, 25.3, 27.2, 27.9, 31.5, 32.2, 34.8, 37.8, 42.9, 47.5, 55.3, 56.5, 69.9, 73.0, 80.3, 127.3, 127.4, 128.2, 170.3, 171.5, 174.4. HRMS calcd for C₃₀H₄₉N₂O₅ (MH⁺): 517.3641. Found (DCI): 517.3613 (Δ=5.5 ppm).

### Synthesis of 3-(R)-(1-(2-(S)-benzyloxymethyl-pyrrolidine-1-carbonyl)-2-(S)-methyl-propylcarbamoyl)-octanoic acid (16)

To a solution of 15 (5.16 g, 10.0 mmol) in CH₂Cl₂ (10 mL) is added TFA (10 mL). The solution is stirred for 2 h and then diluted with CH₂Cl₂ (50 mL). The solution is washed with 50 mL portions of 1 N HCl, water, and brine. It is then dried over Na₂SO₄ and concentrated to give pure 16 as a clear colorless oil (4.19 g, 9.10 mmol, 91%). ¹H NMR (400 MHz, CDCl₃) δ 0.84 (t, J=6.8 Hz, 3 H), 0.91 (d, J=13.1, 3 H), 0.93 (d, J=13.1, 3 H), 1.14-1.29 (m, 6 H), 1.30-1.37 (m, 2 H), 1.39-1.46 (m, 2 H), 1.91-2.15 (m, 4 H), 3.08-3.18 (m, 1 H), 3.56-3.59 (m, 2 H), 3.45 (dd, J=16.8, 6.1 Hz, 1 H), 3.78 (dd, J=16.8, 8.1, 1 H), 3.89-3.92 (m, 1 H); 4.26-4.37 9 (m, 1 H), 4.48 (s, 2 H), 4.57 (t, J=8.7 Hz, 1 H), 7.28-7.40 (m, 5 H); ¹³C NMR (100 MHz, CDCl₃) δ 13.7, 18.2, 18.7, 22.3, 24.2, 26.3, 26.6, 30.9, 31.3, 32.2, 34.1, 36.1, 40.9, 42.2, 48.8, 57.0, 58.1, 69.5, 73.7, 127.4, 127.6, 128.3, 137.2, 171.5, 176.1, 176.8. HRMS calcd for C₂₆H₄₀N₂O₅ (MH⁺): 461.3015. Found (DCI): 461.3023 (Δ=−1.6 ppm).

### Synthesis of 3-(R)-(1-(2-(S)-benzyloxymethyl-pyrrolidine-1-carbonyl)-2-(S)-methyl-propylcarbamoyl)-octanoic acid 2,5-dioxo-pyrrolidin-1-yl ester (17)

To a solution of 16 (4.19 g, 9.10 mmol) and N-hydroxysuccinimide (1.88 g, 16.38 mmol) in 1,4-dioxane (30 mL) is added a solution of DCC (2.472 g, 12 mmol) in 12 mL dioxane. The mixture is stirred overnight and filtered thru celite. The solids are washed with cold acetone and the filtrate concentrated in vacuo. The residue is redissolved in acetone, cooled to 0° C., and filtered. The filtrate is concentrated and the residue taken in ether. The solution is washed with 50 mL portions of 0.5 N HCl, sat. NaHCO₃, water, and brine. It is then dried over Na₂SO₄ and concentrated. The residue is chromatographed in silica gel using hexane/ethyl acetate (1:10) as eluant to yield 17 as a white solid (4.313 g, 7.73 mmol, 85%). ¹H NMR (400 MHz, CDCl₃) δ 0.84 (t, J=6.7 Hz, 3 H), 0.91 (d, J=13.1, 3 H), 0.93 (d, J=13.1, 3 H), 1.05-1.16 (m, 2 H), 1.21-1.30 (m, 2 H), 1.57-1.62 (m, 2 H), 1.64-1.72 (m, 2 H), 1.87-2.10 (m, 4 H), 2.43-2.51 (m 1 H), 2.67-2.79 (m, 2 H), 3.45-3.50 (m, 2 H), 3.55 (dd, J=9.2, 2.8, 1 Hz, 1 H), 3.63 (dd, J=9.2, 5.6 Hz, 1 H), 4.30 (bs, 4 H), 4.48 (s, 2 H); 4.60 (t, J=8.5 Hz, 2 H), 7.27-7.35 (m, 5 H); ¹³C NMR (100 MHz, CDCl₃) δ 13.8, 17.7, 19.2, 22.3, 25.5, 26.5, 26.8, 31.2, 32.4, 33.5, 36.7, 42.7, 47.8, 55.7, 56.8, 69.7, 73.1, 127.3, 127.5, 128.3, 138.2, 167.4, 170.7, 173.2, 175.0, 175.3. HRMS calcd for C₃₀H₄₄N₃O₇ (MH⁺): 558.3179. Found (FAB): 558.3184 (Δ=0.5 ppm).

### Synthesis of N4-benzyloxy-N1-(1-(2-(S)-benzyloxymethyl-pyrrolidine-1-carbonyl)-2-(S)-methyl-propyl)-2-(R)-pentylsuccinamide (19)

A solution of 17 (2.50 g, 4.49 mmol) in DMF (20 mL) is added, via cannula, to a suspension of 18 (1.12 g, 7.0 mmol) in triethylamine (10 mL) and DMF (20 mL). The mixture is stirred overnight afterwhich it is diluted with CH₂Cl₂ (50 mL) and washed with water (50 mL×2), 1 N HCl (50 mL), sat. NaHCO₃ (50 mL), water (50 mL), and brine (50 mL). The solution is dried over Na₂SO₄ and concentrated in vacuo. The residue is chromatographed in silica gel using hexane/ethyl acetate (1:10) as eluant to give 19 as a white solid (2.50 g, 4.42 mmol, 98% yield). ¹H NMR (400 MHz, CDCl₃) δ 0.83 (t, J=6.7 Hz, 3 H), 0.90 (d, J=13.1, 3 H), 0.93 (d, J=13.1, 3 H), 1.05-1.16 (m, 2 H), 1.21-1.30 (m, 2 H), 1.57-1.62 (m, 2 H), 1.64-1.72 (m, 2 H), 1.87-2.10 (m, 4 H), 2.43-2.51 (m 1 H), 2.67-2.79 (m, 2 H), 3.45-3.50 (m, 2 H), 3.55 (dd, J=9.2, 2.8, 1 Hz, 1 H), 3.63 (dd, J=9.2, 5.6 Hz, 1 H), 4.48 (s, 2 H); 4.60 (t, J=8.5 Hz, 2 H), 4.87 (s, 2 H), 7.27-7.37 (m, 10 H); ¹³C NMR (100 MHz, CDCl₃) δ 13.7, 18.2, 18.7, 22.3, 24.2, 26.3, 26.6, 30.9, 31.3, 32.2, 34.1, 36.1, 40.9, 42.2, 48.8, 57.0, 58.1, 69.5, 69.5, 73.7, 127.3, 127.5, 127.6, 128.3, 128.4, 128.5, 128.8 138.2, 171.9, 176.3, 177.2. HRMS calcd for C₃₃H₄₈N₃O₅ (MH⁺): 566.3593. Found (FAB): 566.3573 (Δ=−2.1 ppm).

### Synthesis of (S,S,R)-actinonin (1)

Pd(OH)₂/C (Pearlman's catalyst) (525 g, ˜100 mg Pd content) in a 100 mL two-neck round bottom flask fitted with a stirring bar is activated by repeated evacuation of the flask and introduction of hydrogen gas. Once activated, a solution of 19 (2.50 g, 4.42 mmol) in methanol (50 mL) is added via syringe. The amount of hydrogen consumed is measured and the reaction is monitored closely by TLC. Once the amount of calculated hydrogen gas is exceeded and the starting material totally consumed, the hydrogen is removed and the reaction mixture filtered through celite. The solids are washed with liberal amounts of methanol. The methanol is removed in vacuo and the residue chromatographed in silica gel using CH₂Cl₂/CH₃OH (10:1) as eluant to afford 1 as an off-white solid (3.757 g, 9.74 mmol, 82%). (α)²¹_{D} −51 (c=0.19, CH₃OH); m.p. 141.5-142.0° C., mixed m.p. 141.7-142.4° C., (m.p. of commercial sample (Sigma®) 140.6-141.4° C.) (lit.² 148-149° C.). ¹H NMR (400 MHz, CDCl₃) δ 0.71 (t, J=6.6 Hz, 3 H), 0.95-1.18 (m, 11 H), 1.25-1.46 (m, 2 H), 1.50-1.61 (m, 1 H), 1.51-1.72 (m, 1 H), 1.76-1.95 (m, 2 H), 1.96-2.06 (m, 1 H), 2.22-2.34 (m, 1 H), 2.68 (dd, J=13.8, 5.6 Hz, 1 H), 3.04 (dd, J=13.8, 7.5 Hz, 1 H), 3.51-3.62 (m, 2 H), 3.82-3.91 (m, 1 H), 4.08-4.18 (m, 2 H), 4.52-4.60 (m, 1 H), 4.95 (t, J=8.4 Hz, 1 H); ¹³C NMR (100 MHz, CDCl₃) δ 16.3, 21.1, 21.9, 24.1, 24.9, 26.7, 29.5, 29.9, 33.6, 34.1, 35.5, 39.5, 45.6, 50.3, 59.3, 62.4, 65.5, 171.3, 174.4, 177.8. m/z 386.3 (MH⁺).

### EXAMPLE 5

### Synthesis of Analogs of (S,S,R)-actinoninN4-hydroxy-N1-(1-(2-methyl-pyrrolidine-1-carbonyl)-3-methyl-propyl)-2-pentyl-succinamide (27)

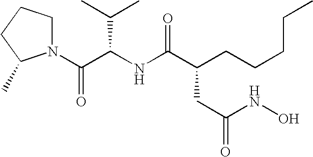

The synthesis is comprised of the following steps: (a) a solution of 2-(S)-methylpyrrolidine hydrobromide 20 in dimethylformamide is treated with triethylamine followed by a solution of 2-tert-butoxycarbonylamino-3-methylbutyric acid 2,5-dioxo-pyrrolidin-1-yl 12 in dimethylformamide to yield (1-(2-methylpyrrolidine-1-carbonyl)-2-methyl-propyl)-carbamic acid tert-butyl ester 21; which is then (b) dissolved in methylene chloride, or any suitable solvent, and treated with trifluoroacetic acid to yield 2-amino-1-(2-methylpyrrolidin-1-yl)-3-methylbutan-1-one 22; (c) a solution of 22 in dimethylformamide is treated with triethylamine followed by a solution of 8 in dimethylformamide to yield 3-(1-(2-(S)-methylpyrrolidine-1-carbonyl)-2-(S)-methylpropyl-carbamoyl)-octanoic acid tert-butyl ester 23; (d) treatment of 23 in dichloromethane with trifluoroacetic acid to yield 3-(1-(2-methyl-pyrrolidine-1-carbonyl)-2-methyl-propylcarbamoyl)-octanoic acid 24; (e) treatment of a solution 24 and hydroxysuccinamide 7 with dicyclohexylcarbodiimide to afford 3-(1-(2-methyl-pyrrolidine-1-carbonyl)-2-methylpropylcarbamoyl)-octanoic acid 2,5-dioxo-pyrrolidin-1-yl ester 25; (f) treatment of a suspension of O-benzylhydroxyamine hydrochloride 18 in dimethylformamide with triethylamine followed by a solution of 25 in dimethylformamide to afford N4-benzyloxy-N1-(1-(2-benzyloxymethyl-pyrrolidine-1-carbonyl)-2-methyl-propyl)-2-pentyl-succinamide 26. (g) hydrogenating 26 with hydrogen gas and palladium in activated carbon wherein N4-hydroxy-N1-(1-(2-methyl-pyrrolidine-1-carbonyl)-3-methyl-propyl)-2-pentyl-succinamide 27 is thereby formed.

### N4-hydroxy-N1-(1-(2-hydroxymethyl-pyrrolidine-1-carbonyl)-3-methyl-butyl)-2-pentyl-succinamide (35)

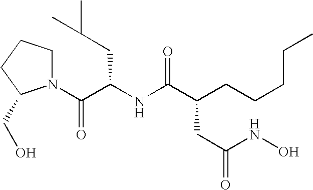

The synthesis is comprised of the following steps: (a) a solution of 2-(S)-benzyloxymethylpyrrolidine 10 in THF is treated with triethylamine followed by a solution of 2-tert-butoxy carbonyl amino-4-methylpentanoic acid 2,5-dioxo-pyrrolidin-1-yl 28 in THF to yield (1-(2-benzyloxymethyl-pyrrolidine-1-carbonyl)-3-methyl-isobutyl)-carbamic acid tert-butyl ester 29; which is then (b) dissolved in methylene chloride and treated with trifluoroacetic acid to yield 2-amino-1-(2-benzyloxymethylpyrrolidin-1-yl)-4-methylpentan-1-one 30; (c) a solution of 30 in dimethylformide or any suitable solvent is treated with triethylamine followed by a solution of 8 in dimethylformamide or any suitable solvent to yield 3-(1-(2-(S)-benzyloxymethylpyrrolidine-1-carbonyl)-2-(S)-methyl isopropyl-carbamoyl)-octanoic acid tert-butyl ester 31; (d) treatment of 31 in dichloromethane with trifluoroacetic acid to yield 3-(1-(2-benzyloxymethyl-pyrrolidine-1-carbonyl)-2-methyl-isobutyl carbamoyl)-octanoic acid 32; (e) treatment of a solution 32 and hydroxysuccinamide with dicyclohexylcarbodiimide or any suitable imide to afford 3-(1-(2-benzyloxymethyl-pyrrolidine-1-carbonyl)-2-methylisocarbamoyl)-octanoic acid 2,5-dioxo-pyrrolidin-1-yl ester 33; (f) treatment of a suspension of O-benzylhydroxyamine hydrochloride 18 in dimethylformamide with triethylamine followed by a solution of 33 in dimethylformamide to afford N4-benzyloxy-N1-(1-(2-benzyloxymethyl-pyrrolidine-1-carbonyl)-2-methyl-isobutyl)-2-pentyl-succinamide 34. (g) hydrogenating 34 with hydrogen gas and palladium hydroxide in activated carbon wherein N4-hydroxy-N1-(1-(2-hydroxymethyl-pyrrolidine-1-carbonyl)-3-methyl-butyl)-2-pentyl-succinamide 35 is thereby formed.

### EXAMPLE 6

### Structure and Nomenclature of Additional Analogs and Derivatives of (S,S,R)-actinonin N4-hydroxy-N1-(1-(2-hydroxymethyl-pyrrolidine-1-carbonyl)-3-methyl-butyl)-2-pentyl succinamide (41)

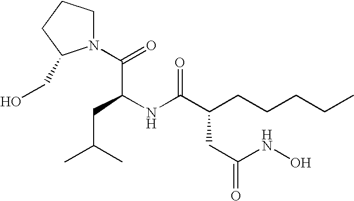

### N1-(1-benzyl-2-(2-hydroxymethyl-pyrrolidin-1-yl)-2-oxo-ethyl)-N4-hydroxy-2-pentyl-succinamide (42)

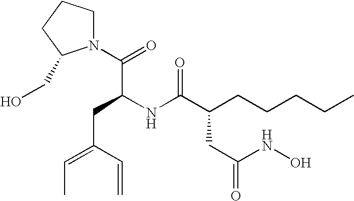

### N4-hydroxy-N1-(1-(4-hydroxy-benzyl)-2-(2-hydroxymethyl-pyrrolidin-1-yl)-2-oxo-ethyl)-2-pentyl-succinamide (43)

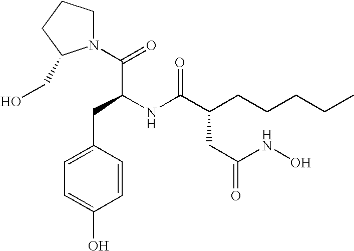

### N4-hydroxy-N1-(2-(2-hydroxymethyl-pyrrolidin-1-yl)-1(1H-indol-3-yl-methyl)-2-oxo-ethyl)-2-pentyl-succinamide (44)

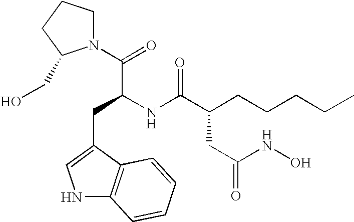

### N1-(5-amino-1-(2-hydroxymethyl-pyrrolidine-1-carbonyl)-pentyl)-N4-hydroxy-2-pentyl-succinamide (45)

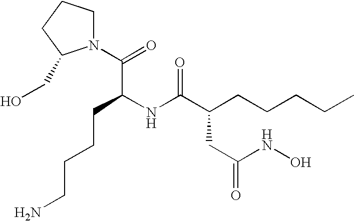

### N4-hydroxy-N1-(1-(2-hydroxymethyl-piperidine-1-carbonyl)-2-methyl-propyl)-2-pentyl-succinamide (46)

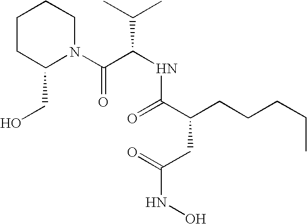

### N4-hydroxy-N1-(1-(2-hydroxycarbamoyl-pyrrolidine-1-carbonyl)-3-methyl-butyl)-2-pentyl succinamide (47)

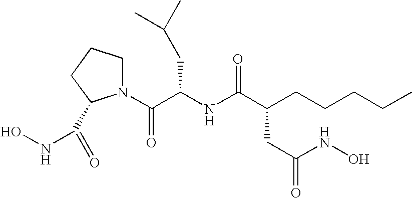

### N4-hydroxy-N1-(1-(2-hydroxymethyl-pyrrolidine-1-carbonyl)-2-methyl-propyl)-2-methyl-succinamide (48)

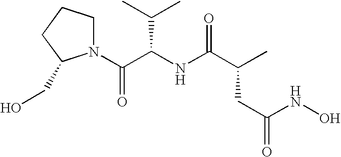

### N1-(1-(2-hydroxymethyl-pyrrolidine-1-carbonyl)-3-methyl-butyl)-2-pentyl-succinamide (49)

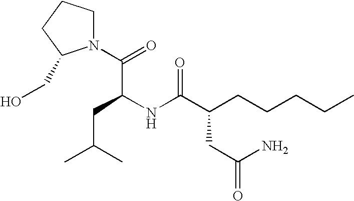

### N1-(1-benzyl-2-(2-hydroxymethyl-pyrrolidin-1-yl)-2-oxo-ethyl)-2-pentyl-succinamide (50)

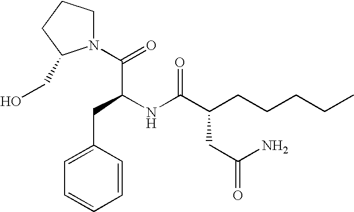

### N1-(1-(2-methyl-pyrrolidine-1-carbonyl)-2-methyl-propyl)-2-pentyl-succinamide (51)

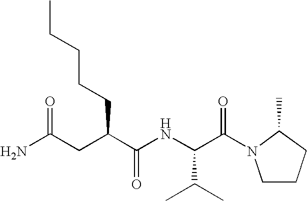

### N4-hydroxy-N1-(1-benzyl-2-(2-methyl-pyrrolidin-1-yl)-2-oxo-ethyl-2-pentyl-succinamide (52)

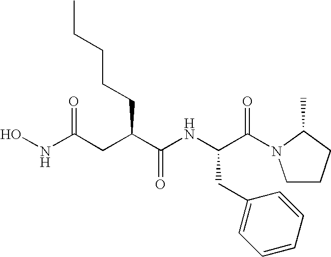

### N4-hydroxy-N1-(1-(2-methylamine-pyrrolidine-1-carbonyl)-2-methyl-propyl)-2-pentyl-succinamide (53)

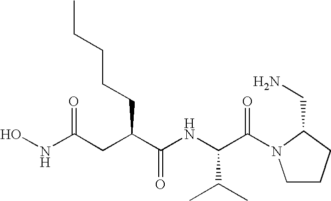

### 3-[1-(2-hydroxymethyl-pyrrolidin-1-yl)-2-benzylcarbamoyl]-octanoic acid (54)

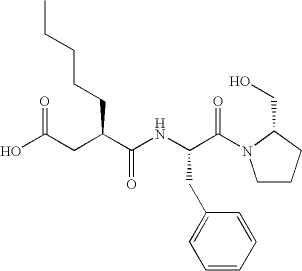

### N4-hydroxy-N1-(1-(methyl-2-carboxy-pyrrolidine-1-carbonyl)-2-methyl-propyl)-2-pentyl-succinamide (55)

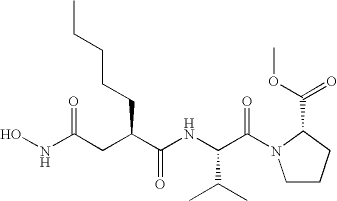

### N4-hydroxy-N1-(1-(2-carboxy-pyrrolidine-1-carbonyl)-2-methyl-propyl)-2-pentyl-succinamide (56)

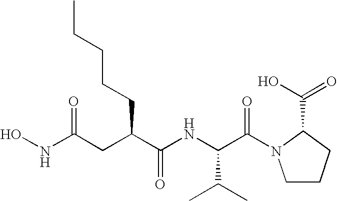

### N4,N4-diethyl-N1-(1-benzyl-2-(2-hydroxymethyl-pyrrolidin-1-yl)-2-oxo-ethyl-2-pentyl succinamide (57)

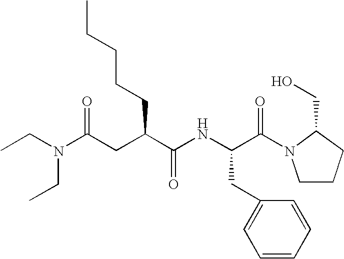

### N4-ethyl-N1-(1-benzyl-2-(2-hydroxymethyl-pyrrolidin-1-yl)-2-oxo-ethyl-2-pentyl-succinamide (58)

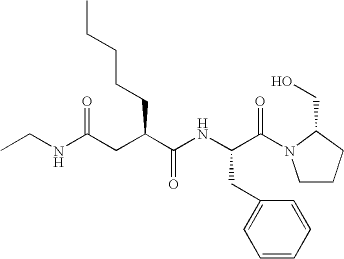

### N4-(2,4-methoxybenzyl)-N1-(1-benzyl-2-(2-hydroxymethyl-pyrrolidin-1-yl)-2-oxo-ethyl-2-pentyl-succinamide (59)

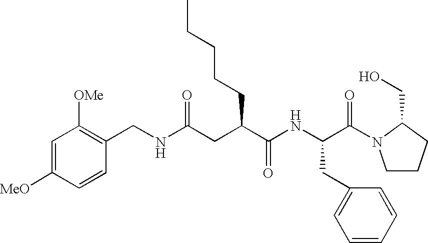

### 2-(N′,N′-dimethyl-hydrazinocarbonylmethyl)-heptanoic acid [1-benzyl-2-(2-hydroxymethyl-pyrrolidin-1-yl)-2-oxo-ethyl]-amide (60)

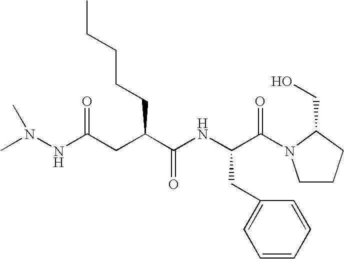

### N4-(4-nitrobenzyl)-N1-(1-benzyl-2-(2-hydroxymethyl-pyrrolidin-1-yl)-2-oxo-ethyl-2-pentyl-succinamide (61)

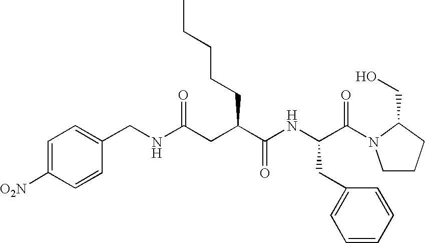

### 2-[2-(4-methyl-piperazin-1-yl)-2-oxo-ethyl]-heptanoic acid [1-benzyl-2-(2-hydroxymethyl-pyrrolidin-1-yl)-2-oxo-ethyl]-amide (62)

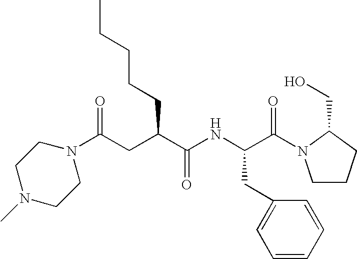

### N4-(methoxy)-N1-(1-benzyl-2-(2-hydroxymethyl-pyrrolidin-1-yl)-2-oxo-ethyl-2-pentyl-succinamide (63)

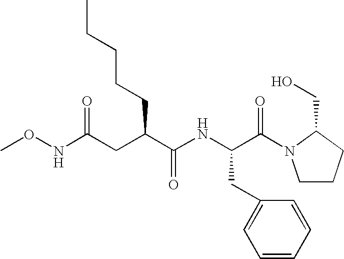

### N4-(piperidin-1-carbonyl)-N1-(1-benzyl-2-(2-hydroxymethyl-pyrrolidin-1-yl)-2-oxo-ethyl-2-pentyl-succinamide (64)

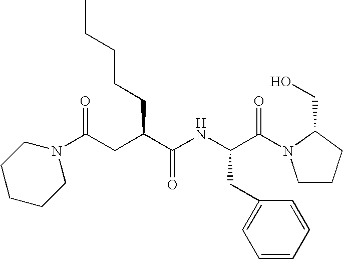

### N4,N4-methoxymethyl-N1-(1-benzyl-2-(2-hydroxymethyl-pyrrolidin-1-yl)-2-oxo-ethyl-2-pentyl-succinamide (65)

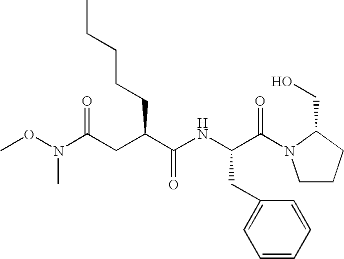

### N4-hydroxy-N1-(2-oxo-2-(piperidin-1-yl)ethyl)-2-pentyl succinamide (66)

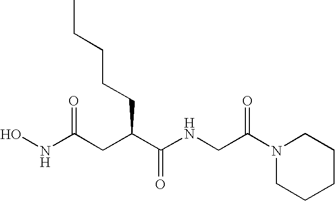

Compound 66 is synthesized from the coupling with protected glycine followed by subsequent coupling with piperidine.

### 4-((2-(2(hydroxyamino)-2-oxoethyl)heptanamido)-5-oxo-5-(piperidin-1-yl)pentanoic acid (67)

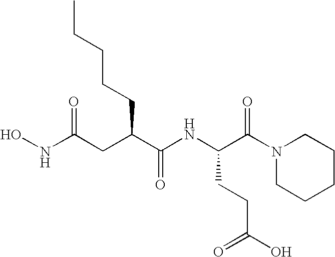

Compound 67 is synthesized from the coupling with protected glutamic acid ollowed by deprotection and subsequent the coupling with piperidine.

### N4-hydroxy-N1-(1-piperidine-1-carbonyl)-pentyl)-2-pentyl succinamide (68)

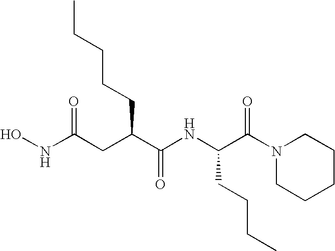

Compound 68 is synthesized from the coupling of protected proline followed by deprotection and subsequent the coupling with piperidine.

### N4-hydroxy-N1,N1-(1-diethyl-1-carbonyl)-2-methyl-propyl)-2-pentyl succinamide (69)

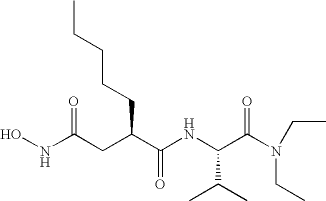

### N4-hydroxy-N1-(1-piperidine-1-carbonyl)-2-methyl-propyl)-2-pentyl succinamide (70)

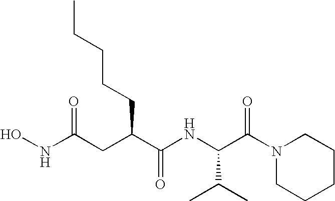

### N4-hydroxy-N1-(1-morpholine-1-carbonyl)-2-methyl-propyl))-2-pentyl succinamide (71)

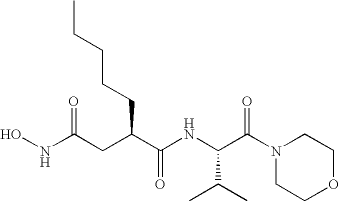

### N-hydroxy-3(2-(piperidine-1-carbonyl)pyrrolidine-1-carbonyl)octanamide (72)

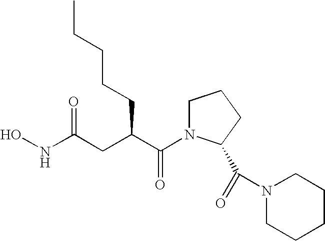

Compound 72 is synthesized using proline for R² and piperidine for R¹ according to the general structure. Its synthesis is the same as described herein except for the substitution of proline instead of valine and piperidine instead of prolinol.

### (5R,8S,12aS)-8-benzyl-5-pentyloctahydropyrrolo[2,1-c][1,4,7]oxadiazacyclundecine-3,6,9(1H)-trione (73)

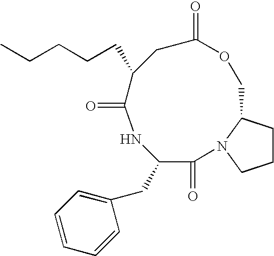

Compound 73 was derived from the DCC cyclization of the free acid-prolinol analog.

### N4-hydroxy-N1-butyl-2-pentylsuccinamide (74)

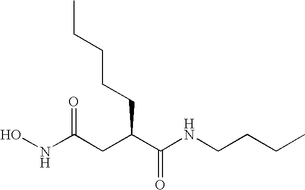

Compound 74 is synthesized from the first intermediate by coupling the acid with butylamine followed by deprotection of the tert-butyl ester with TFA, and subsequent coupling with benzylhydroxylamine and hydrogenation.

### N-hydroxy-3-(4-isopropyl-2-oxooxazolidine-3-carbonyl)octanamide (75)

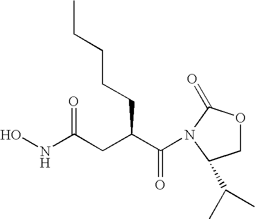

Compound 75 was derived from the intermediate in which the tert-butyl ester was deprotected with TFA and subsequently coupled with benzylhydroxylamine and hydrogenated, all steps having been previously described.

### EXAMPLE 7

### Cytotoxicity and Anti-tumor Activity of Actinonin

The therapeutic use of the compounds of the present invention in treatment of neoplastic diseases is illustrated. Actinonin was assayed for cytotoxity in human ovarian carcinoma, prostate carcinoma, mammary carcinoma, head and neck squamous cell carcinoma (HNSSC), non-small-cell-lung-cancer adenocarcinoma (NSCLC-AdCa), and non-small-cell-lung-cancer squamous cells (NSCLC-SSC) (15). Actinonin was also tested against acute mylegenous leukemia (AML) cells. Cells were grown for 5 days ± varying concentrations of actinonin. Cell number were determined by XTT assay on an automatic plate reader. The results are shown in Table 2. Actinonin is remarkably cytotoxic in the μM range against all the cell lines tested. These growth inhibitory properties provide for the use of these compounds as anti-tumor agents or for the use in the treatment of neoplastic diseases.

TABLE 2
Summary of cell culture data with actinonin^{a}

| Cell line | Histology | IC₅₀ (μM ± SEM) | n Value |
|---|---|---|---|
| A2780 | Ovarian Ca | 4.8 ± 1 | 3 |
| TSU-PR¹ | Prostate Ca | 9.1 ± 1.5 | 3 |
| PC-3 | Prostate Ca | 10.0 ± 1 | 3 |
| DU-145 | Prostate Ca | 17.03 | 3 |
| HL-60 | PML (CML?) | 6.8 ± 1 | 4 |
| MDA- | Mammary Ca | 7.1 ± 1 | 4 |
| MB468 | | | |
| SK-BRIII | Mammary Ca | 7.9 ± 1 | 3 |
| HT1080 | HNSSC | 11.0 ± 2 | 4 |
| SK-LC-8 | NSCLC-AdCa | 14.0 ± 2 | 3 |
| SK-LC-16 | NSCLC-SSC | 12.0 ± 2 | 3 |

Actinonin was also evaluated against the CWR22 human prostate tumor xenografted in nude mice (15). The results are summarized in Table 3. Actinonin shows excellent tumor growth inhibition at a dose slightly below the maximum tolerated dose (MTD) for the mouse.

TABLE 3
Anti-tumor activity of actinonin against the CWR22 human
prostate tumor in nude mice.
Initial tumor diameter = 4.4 + 0.3 min (45 mm³)

| | Change in | Ave. Tumor | Change in | |
|---|---|---|---|---|
| Rₓ | weight | Diameter | tumor Vol. | Inhibition |
| (mg/kg) | (%) | (mm ± SEM) | (mm³) | T/C % |
| — | | 13.3 ± 2 | +1190 | |
| 300 ip | −2 | 5.4 ± 1 | +36 | 97 |
| 600 ip | −3 | 7.1 ± 2 | +142 | 88 |

### EXAMPLE 8

### Evaluation of Actinonin and Its Analogs Against Daudi Lymphomic and HL-60 Leukemic Cells

Actinonin was purchased from Sigma (St. Louis, Mo.). Biotinylated-Actinonin and all analogs were synthesized. Actinonin stock solution was 5 mg/ml in 10% ethanol. Biotinylated-actinonin and all other analogs were diluted in 10% DMSO to give stock solutions in the range of 1-20 mg/ml.

### Cell Lines and Culture Conditions

Daudi (B lineage Burkitt's lymphoma, CD13 negative) and HL60 (acute myeloid leukemia, CD13 positive) cells were maintained in culture using RPMI 1640 supplemented with 10% heat-inactivated FBS (Omega Scientific, Inc., Tarzana, Calif.) and 1% L-glutamine (Gibco/Invitrogen, Carlsbad, Calif.) at 37° C. in a humidified atmosphere of 5% CO₂. Cell viability was higher than 90%, and cells were free of mycoplasma contamination.

### Inhibition of Tritiated Thymidine Incorporation

An aliquot of 200 ul of cells (10,000 cells/well) was washed and incubated at 37° C. in 96-well plates in the presence or absence of actinonin/analogs. Serial dilutions were made in complete media. After 5 days of incubation, 50 ul of 10 uCi/mL tritiated thymidine (PerkinElmer, Boston, Mass.) was added to each well and allowed to incorporate for 5 hours. Plates were frozen at −80° C. overnight and cells were harvested onto filtermats (Wallac, Finland) using a semi-automatic harvester (Skatron, Sterling, Va.). Filtermats were counted in a 1205 Betaplate™ liquid scintillation counter (Wallac, Finland). Tables 4 and 5 show the results of the actinonin analogs on cell viability. The reference numbers refer to those analogs disclosed in Example 6. Compounds 35, 42, 48, 52, 53, and 56 are effective at inhibiting cell growth.

TABLE 4

5 day thymidine incorporation

| | Daudi | HL60 |
|---|---|---|
| Compound | IC50 (ug/mL) | IC50 (ug/mL) |
| Actinonin | 2.6 | 4.1 |
| Biotin-Actinonin | >100 | >100 |
| #35 | 5.7 | 7.1 |
| #49 | >50 | >50 |
| #51 | 6.6 | 6.9 |
| #42 | 5.8 | 7.1 |
| #48 | 8.0 | 10.5 |
| #52 | 1.2 | 5.2 |
| #53 | 7.0 | 10.0 |
| Biotin-Act | 20.0 | 50.0 |
| #54 | ND | >100 |
| #57 | ND | >100 |
| #58 | ND | >100 |
| #59 | ND | >100 |
| #60 | ND | 90.0 |
| #61 | ND | >100 |
| #62 | ND | >100 |
| #63 | ND | >100 |
| #64 | ND | >100 |
| #65 | ND | >100 |
| Calpeptin | 6.0 | 50.0 |
| DL-Thiorphan | >100 | >100 |
| #55 | 0.4016 | 2.773 |
| #56 | >100 | >100 |

TABLE 5

5 day thymidine incorporation

| | Daudi | HL60 |
|---|---|---|
| Compound | IC50 (uM) | IC50 (uM) |
| Actinonin | 6.7 | 10.6 |
| Biotin-Actinonin | >138 | >138 |
| #35 | 14.1 | 17.7 |
| #49 | >130 | >130 |
| #51 | 18.7 | 19.5 |
| #42 | 12.9 | 15.8 |
| #48 | 22.4 | 29.5 |
| #52 | 2.7 | 12.1 |
| #53 | 18.2 | 26.0 |
| Biotin-Act | 27.6 | 69.1 |
| #54 | ND | >239 |
| #57 | ND | >211 |
| #58 | ND | >225 |
| #59 | ND | >176 |
| #60 | ND | 195.5 |
| #61 | ND | >181 |
| #62 | ND | >200 |
| #63 | ND | >224 |
| #64 | ND | >206 |
| #65 | ND | >217 |
| Calpeptin | 16.6 | 137.9 |
| DL-Thiorphan | >395 | >395 |
| #55 | 0.97 | 6.7 |
| #56 | >251 | >251 |

Corrected concentrations for cuvette dilution (1/50) in PDF assay

The following references are cited herein:
1. Gordon, et al., Nature, Vol. 195, pg. 701 (1962).
2. Tieku, et al., Biological Pharmacology 1992, 44, 1725.
3. Fujii, et al., Biol. Pharm. Bull., 1996, 19, 6.
4. Xu, et al. Clinical Cancer Research, 1998, 4, 171.
5. Sayama, et al., Cancer Letters, 1995, 171.
6. Bouboutou, et al. in Second Forum on Peptides, Eds. A. Aubry, M. Marraud, B. Vitoux. Collogue INSERM: John Libbey Eurotext Ltd., 1989, 174, 341.
7. Harper, E. Ann. Rev. Biochem, 1980, 49, 1063.
8. Gordon, et al., J. Chem. Soc. Perkin Trans 1. 1975, 819-824.
9. Umezawa, et al., J. Antibiotics, 1985, 38, 1629-1630.
10. Faucher, et al., J. Antibiotics, 1987, 40, 1757-1761.
11. Anderson, et al., J. Chem. Soc. Perkin Trans 1. 1975, 825-830.
12. Bashiardes, et a. J. Chem. Soc. Perkin Trans. 1, 1993, 459-469.
13. Evans, et al., J. Am. Chem. Soc. 1982, 104, 1737.
14. Gage, J. R., Evans, D. A. Org, Synth. 1989, 68, 83.
15. Sirotnak, et al. Cancer Chemother. Pharmacol 1998, 42, 313.
16. Broek, et al. J. Org. Chem., 1984, 49, 1691-1695.
17. Giglione, et al. The EMBO Journal 2000, 19(21):5916-29.
18. Dirk, et al. Plant Physiology, 2001, 127:97-107.
19. Bracchi-Ricard, et al. Archives of Biochemistry and Biophysics, 2001, 396(2):162-170.

Any patents or publications mentioned in this specification are indicative of the levels of those skilled in the art to which the invention pertains. Further, these patents and publications are incorporated by reference herein to the same extent as if each individual publication was indicated to be incorporated specifically and individually by reference.

One skilled in the art will readily appreciate that the present invention is well adapted to carry out the objects and obtain the ends and advantages mentioned, as well as those inherent therein. The present examples along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

1. An analog or derivative compound of (S,S,R)-(−)-actinonin having the structure: 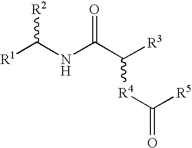
wherein R¹ is hydrogen, C(O)R⁶ or R¹ in combination with N is 2-oxomorpholine;
R² is hydrogen, methyl, CH₂CH(CH₃)₂, (CH₂)₂CH₃, CH(CH₃)₂, (CH₂)₃CH₃, (CH₂)₄NH₂, (CH₂)₃CO₂H, phenyl, 3,4-dichlorophenyl, biphenyl, benzyl, 4-hydroxybenzyl, piperidine, N-Boc-4-piperidine, CH₂-(N-Boc-4-piperidine), 4-tetrahydropyran, CH₂-4-tetrahydropyran, 3-methyl indolyl, 2-naphthyl, 3-pyridyl, 4-pyridyl, 3-thienyl;
R³ is R² or C₃₋₈alkyl,
R⁴ is C₁₋₃alkyl;
R⁵ is NH₂, OH, NHOH, NHOCH₃, N(CH₃)OH, N(CH₃)OCH₃, NHCH₂CH₃, NH(CH₂CH₃), NHCH₂(2,4-(OCH3)₂Ph, NHCH₂(4-NO₂)Ph, NHN(CH₃)₂, proline, or 2-hydroxymethyl pyrrolidine; and
R⁶ is an optionally substituted or halogenated alkyl, aryl, heteroalkyl or heteroaryl amine, said R⁶ further comprising a cyclic or bicyclic structure; or
pharmaceutically acceptable salts or hydrates thereof.

2. The compound of claim 1, wherein R¹ is hydrogen, R² is (CH₂)₃CH₃, R³ is pentyl, R⁴ is methylene, and R⁵ is NHOH.

3. The chemical of claim 2, wherein said compound is N4-hydroxy-N1-butyl-2-pentylsuccinamide.

4. The compound of claim 1, wherein R¹ in combination with N is 2-oxomorpholine, R² is CH(CH₃)₂, R³ is pentyl, R⁴ is methylene, and R⁵ is NHOH.

5. The compound of claim 4, wherein said compound is N-hydroxy-3-(4-isopropyl-2-oxooxazolidine-3-carbonyl)octanamide.

6. The compound of claim 1, having the structure: 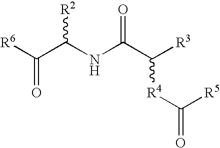
R² is hydrogen, methyl, CH₂CH(CH₃)₂, (CH₂)₂CH₃, CH(CH₃)₂, (CH₂)₃CH₃, (CH₂)₄NH₂, (CH₂)₃CO₂H, phenyl, 3,4-dichlorophenyl, biphenyl, benzyl, 4-hydroxybenzyl, piperidine, N-Boc-4-piperidine, CH₂-(N-Boc-4-piperidine), 4-tetrahydropyran, CH₂-4-tetrahydropyran, 3-methyl indolyl, 2-naphthyl, 3-pyridyl, 4-pyridyl, 3-thienyl;
R³ is R² or C₃₋₈alkyl,
R⁴ is C₁₋₃alkyl;
R⁵ is NH₂, OH, NHOH, NHOCH₃, N(CH₃)OH, N(CH₃)OCH₃, NHCH₂CH₃, NH(CH₂CH₃), NHCH₂(2,4-(OCH3)₂Ph, NHCH₂(4-NO₂)Ph, NHN(CH₃)₂, proline, or 2-hydroxymethyl pyrrolidine; and
R⁶ is NHCH₂Ph, NHCH₃, NHCH₂CH₃, N(CH₃)₂, N(CH₂CH₃)₂, NHCH₂(2,4-(OCH₃)₂Ph, NHCH₂(4-NO₂Ph), hexamethyleneamine, hexamethyleneimine, methyl 2- or 3-hexamethyleneamine carboxylate, heptamethyleneamine, pyrrole, indole, aziradine, imidazole, 1,4-dioxan-2-yl-methylamine, 3,4-dihydro-2H-1,4-benzoxazin-6-ol, 6-methoxy-1,2,3,4-tetrahydro-isoquinoline, piperazin-1-yl-pyridin-3-yl-methanone or further comprising:
pyrrolidine optionally substituted with 2-methylamino, 2-hydroxycarbamoyl, one of 2- or 3-hydroxymethyl, one of 2- or 3-methyl, ethyl, benzyl or phenyl, one of 2,3-, 2,4-, or 2,5-dimethyl, 2,5-diethyl, one of methyl-, ethyl-, t-butyl- or benzyl-3-carboxylate, or methyl-(2-methyl-5-carboxylate);
piperidine optionally substituted with 2- or 3-methyl or ethyl, one of methyl-, ethyl-, or benzyl-2-, 3-, 4-carboxylate;
piperazine optionally substituted with 1-benzyl, N-t-boc, 1-furfuryl, 1-isonicotinoyl, or -one of pyridin-2-, 3- or 4-ylmethyl;
morpholine optionally substituted with one of methyl-, ethyl-, or benzyl-2- or 3-carboxylate;
indoline optionally substituted with one of C2-C7 fluoro or methyl-2-carboxylate;
proline optionally substituted to independently form a methyl, ethyl, benzyl or t-butyl ester;
azetidine optionally substituted with one of 2- or 3-methyl or ethyl or a methyl-, ethyl- or benzyl-2- or 3-carboxylate.

7. The compound of claim 6, wherein R² is hydrogen, (CH₂)₃CH₃, or (CH₂)₃CO₂H, R³ is pentyl, R⁴ is methylene, R⁵ is NHOH and R⁶ is piperidine,.

8. The compound of claim 7, wherein said compound is N4-hydroxy-N1-(2-oxo-2-(piperidin-1-yl)ethyl)-2-pentyl succinamide, 4-((2-(2(hydroxyamino)-2-oxoethyl)heptanamido)-5-oxo-5-(piperidin-1-yl) pentanoic acid or N4-hydroxy-N1-(1-piperidine-1-carbonyl)-pentyl)-2-pentyl succinamide.

9. The compound of claim 6, wherein R² is CH(CH₃)₂, R³ is pentyl, R⁴ is methylene, R⁵ is NHOH and R⁶ is morpholine, hexamethyleneimine, or NH(CH₂CH₃).

10. The compound of claim 9, wherein said compound is N4-hydroxy-N1,N1-(1-diethylamino-1-carbonyl)-2-methyl-propyl)-2-pentyl succinamide, N4-hydroxy-N1-(1-piperidine-1-carbonyl)-2-methyl-propyl)-2-pentyl succinamide or N4-hydroxy-N1-(1-morpholine-1-carbonyl)-2-methyl-propyl))-2-pentyl succinamide.

11. The compound of claim 6, wherein R² in combination with N is pyrrolidine.

12. The compound of claim 11, wherein said compound is N-hydroxy-3(2-(piperidine-1-carbonyl)pyrrolidine-1-carbonyl)octanamide.

13. The compound of claim 12, wherein R⁵ and R⁶ combine to form a ring.

14. The compound of claim 13, wherein said compound is (5R,8S, 12aS)-8-benzyl-5-pentyloctahydropyrrolo[2,1-c][1,4,7]oxadiazacyclundecine-3,6,9(1H)-trione.

15. A method for asymmetrically synthesizing a chemical compound having the structure of claim 6, comprising the steps of:
a) forming an optionally O-protected R⁶-1-carbonyl-C2-(R²)-methyleneamine from R⁶ and an N-protected, optionally O-protected, R²-amino acid 2,5-dioxo-pyrrolidinyl ester and deprotecting said N-protected R²-amino acid with a suitable agent comprising trifluoroacetic acid;
b) forming an R³-carbonyl-oxazolidone from 4-isopropyl-oxazolidin-2-one and R³-carbonyl chloride;
c) treating a solution of 4-(S)-isopropyl-oxazolidin-2-one with a solution of a base comprising n-butyl lithium in hexanes and adding an R³-carbonyl chloride thereby forming an R³-carbonyl oxazolidinone;
d) treating a solution of the R³-carbonyl oxazolidinone sequentially with a base comprising lithium diisopropylamide and with a bromo-R⁴ acid-tert-butyl ester thereby forming an oxazolidine-R³-carbonyl-R⁴-acid tert-butyl ester;
e) treating a mixture of the an oxazolidine-R³-carbonyl-R⁴-acid tert-butyl ester in tetrahydrofuran and water sequentially with hydrogen peroxide in water and with lithium hydroxide in water thereby forming a C2(R³)—R⁴-dicarboxylic acid tert-butyl ester;
f) treating a mixture of the C2(R³)—R⁴-dicarboxylic acid 4-tert-butyl ester and hydroxysuccinimide in a solvent comprising dioxane or dimethylformamide with an imide comprising dicyclohexylcarbodiimide thereby forming an C2(R³)—R⁴-dicarboxylic acid tert-butyl ester-(2,5-dioxo-pyrrolidin-1-yl) ester.
g) treating a solution of said optionally O-protected R⁶-1-carbonyl-2-(R²)-methyleneamine in a solvent comprising tetrahydrofuran sequentially with triethylamine and with the C2(R³)—R⁴-dicarboxylic acid tert-butyl ester-(2,5-dioxo-pyrrolidin-1-yl) ester thereby forming an optionally O-protected R⁶-1-carbonyl-2-(R²)-carbamoyl-methylene(R³)—R⁴-carboxylic acid tert-butyl ester;
h) treating a solution of said optionally O-protected R⁶-1-carbonyl-C2(R²)-carbamoyl-methylene(R³)—R⁴-carboxylic acid tert-butyl ester in a solvent comprising methylene chloride with trifluoroacetic acid thereby forming an optionally O-protected R¹-1-carbonyl-C2(R²)-carbamoyl-methylene(R³)—R⁴-carboxylic acid;
i) treating said optionally O-protected R¹-1-carbonyl-2-(R²)-carbamoyl-methylene(R³)—R⁴-carboxylic acid and hydroxysuccinamide with an imide comprising dicyclohexylcarbodiimide thereby forming a optionally O-protected R⁶-1-carbonyl-C2(R²)-carbamoyl-methylene(R³)—R⁴-carboxylic acid 2,5-dioxo-pyrrolidin-1-yl ester;
j) treating a suspension of an optionally O-protected R⁵ or the chloride thereof in a solvent comprising dimethylformamide sequentially with triethylamine and with a solution of said O-protected R⁶-1-carbonyl-C2(R²)-carbamoyl-methylene(R³)—R⁴-carboxylic acid 2,5-dioxo-pyrrolidin-1-yl ester in a solvent comprising dimethylformamide thereby forming an R⁶-1-carbonyl-C2(R²)-carbamoyl-methylene(R³)—R⁴-carbonyl-R⁵, said R⁶ and R⁵ independently optionally O-protected; and
k) hydrogenating said R⁶ and R⁵, said R⁶ and R⁵ independently comprising an O-protecting group, with hydrogen gas and a catalyst comprising palladium hydroxide in activated carbon wherein said chemical compound is thereby formed.

16. The method of claim 15, wherein
R² is hydrogen, CH₂CH₃, (CH₂)₃CH₃, CH(CH₃)₂, or (CH₂)₃CO₂H;
R³ is pentyl;
R⁴ is methylene;
R⁵ is NH₂, OH, NHOH, NHOCH₃, N(CH₃)OH, N(CH₃)OCH₃, NHCH₂CH₃, NH(CH₂CH₃), NHCH₂(2,4-(OCH3)₂Ph, NHCH₂(4-NO₂)Ph, NHN(CH₃)₂, proline, 2-hydroxymethyl pyrrolidine. piperidine or 1-methyl-piperazine; and
R⁶ is piperidine, morpholine, hexamethyleneimine, or NH(CH₂CH₃).

17. The method of claim 16, wherein the compound is N4-hydroxy-N1-(2-oxo-2-(piperidin-1-yl)ethyl)-2-pentyl succinamide, 4-((2-(2(hydroxyamino)-2-oxoethyl)heptanamido)-5-oxo-5-(piperidin-1-yl) pentanoic acid, N4-hydroxy-N1-(1-piperidine-1-carbonyl)-pentyl)-2-pentyl succinamide, N4-hydroxy-N1,N1-(1-diethylamino-1-carbonyl)-2-methyl-propyl)-2-pentyl succinamide, N4-hydroxy-N1-(1-piperidine-1-carbonyl)-2-methyl-propyl)-2-pentyl succinamide, N4-hydroxy-N1-(1-morpholine-1-carbonyl)-2-methyl-propyl))-2-pentyl succinamide, N-hydroxy-3(2-(piperidine-1-carbonyl)pyrrolidine-1-carbonyl) octanamide, (5R,8S,12aS)-8-benzyl-5-pentyloctahydropyrrolo[2,1-c][1,4,7]oxadiazacyclundecine-3,6,9(1H)-trione.

18. A method for treating a neoplastic disease in an individual, comprising administering a pharmacologically effective dose of the compound of claim 1 or of (S,S,R)-(−)-actinonin.

19. The method of claim 18, wherein said chemical compound is N4-hydroxy-N1-(1-(2-hydroxymethyl-pyrrolidine-1-carbonyl)-3-methyl-butyl)-2-pentyl-succinamide, N1-(1-(2-methyl-pyrrolidine-1-carbonyl)-2-methyl-propyl)-2-pentyl-succinamide, N1-(1-benzyl-2-(2-hydroxymethyl-pyrrolidin-1-yl)-2-oxo-ethyl)-N4-hydroxy-2-pentyl-succinamide, N4-hydroxy-N1-(1-(2-hydroxymethyl-pyrrolidine-1-carbonyl)-2-methyl-propyl)-2-methyl-succinamide, N4-hydroxy-N1-(1-benzyl-2-(2-methyl-pyrrolidin-1-yl)-2-oxo-ethyl-2-pentyl-succinamide, N4-hydroxy-N1-(1-(methyl-2-carboxy-pyrrolidine-1-carbonyl)-2-methyl-propyl)-2-pentyl-succinamide, N4-hydroxy-N1-(2-oxo-2-(piperidin-1-yl)ethyl)-2-pentyl succinamide, 4-((2-(2(hydroxyamino)-2-oxoethyl)heptanamido)-5-oxo-5-(piperidin-1-yl) pentanoic acid, N4-hydroxy-N1-(1-piperidine-1-carbonyl)-pentyl)-2-pentyl succinamide, N4-hydroxy-N1,N1-(1-diethylamino-1-carbonyl)-2-methyl-propyl)-2-pentyl succinamide, N4-hydroxy-N1-(1-piperidine-1-carbonyl)-2-methyl-propyl)-2-pentyl succinamide, N4-hydroxy-N1-(1-morpholine-1-carbonyl)-2-methyl-propyl))-2-pentyl succinamide, N-hydroxy-3(2-(piperidine-1-carbonyl)pyrrolidine-1-carbonyl)octanamide, (5R,8S,12aS)-8-benzyl-5-pentyloctahydropyrrolo[2,1-c][1,4,7]oxadiazacyclundecine-3,6,9(1H)-trione, N4-hydroxy-N1-butyl-2-pentylsuccinamide, or N-hydroxy-3-(4-isopropyl-2-oxooxazolidine-3-carbonyl)octanamide.

20. The method of claim 18, wherein said neoplastic disease is a human ovarian carcinoma, a prostate carcinoma, a mammary carcinoma, a head and neck squamous cell carcinoma, a non-small-cell-lung-cancer adenocarcinoma, non-small-cell-lung-cancer squamous cell carcinoma, or acute myologenous leukemia.

21. A method of inhibiting the growth of a tumor cell comprising the step of contacting said cell with a pharmacologically effective amount of the compound of claim 1 or of (S,S,R)-(−)-actinonin.

22. The method of claim 21, wherein said chemical compound is N4-hydroxy-N1-(1-(2-hydroxymethyl-pyrrolidine-1-carbonyl)-3-methyl-butyl)-2-pentyl-succinamide, N1-(1-(2-methyl-pyrrolidine-1-carbonyl)-2-methyl-propyl)-2-pentyl-succinamide, N1-(1-benzyl-2-(2-hydroxymethyl-pyrrolidin-1-yl)-2-oxo-ethyl)-N4-hydroxy-2-pentyl-succinamide, N4-hydroxy-N1-(1-(2-hydroxymethyl-pyrrolidine-1-carbonyl)-2-methyl-propyl)-2-methyl-succinamide, N4-hydroxy-N1-(1-benzyl-2-(2-methyl-pyrrolidin-1-yl)-2-oxo-ethyl-2-pentyl-succinamide, N4-hydroxy-N1-(1-(methyl-2-carboxy-pyrrolidine-1-carbonyl)-2-methyl-propyl)-2-pentyl-succinamide, N4-hydroxy-N1-(2-oxo-2-(piperidin-1-yl)ethyl)-2-pentyl succinamide, 4-((2-(2(hydroxyamino)-2-oxoethyl)heptanamido)-5-oxo-5-(piperidin-1-yl) pentanoic acid, N4-hydroxy-N1-(1-piperidine-1-carbonyl)-pentyl)-2-pentyl succinamide, N4-hydroxy-N1,N1-(1-diethylamino-1-carbonyl)-2-methyl-propyl)-2-pentyl succinamide, N4-hydroxy-N1-(1-piperidine-1-carbonyl)-2-methyl-propyl)-2-pentyl succinamide, N4-hydroxy-N1-(1-morpholine-1-carbonyl)-2-methyl-propyl))-2-pentyl succinamide, N-hydroxy-3(2-(piperidine-1-carbonyl)pyrrolidine-1-carbonyl)octanamide, (5R,8S,12aS)-8-benzyl-5-pentyloctahydropyrrolo[2,1-c][1,4,7]oxadiazacyclundecine-3,6,9(1H)-trione, N4-hydroxy-N1-butyl-2-pentylsuccinamide, or N-hydroxy-3-(4-isopropyl-2-oxooxazolidine-3-carbonyl)octanamide.

23. The method of claim 20, wherein said tumor cell is a human ovarian cancer cell, a prostate cancer cell, a mammary cancer cell, a head and neck squamous cancer cell, a non-small-cell-lung-cancer cell, an adenocarcinoma cell, a non-small-cell-lung-cancer squamous cell, or an acute myologenous leukemic cell.
